(19) United States
(12) Patent Application Publication
(10) Pub. No.: US 2009/0259316 A1
(43) Pub. Date: Oct. 15, 2009

(54) Spacer Devices and Systems for the Treatment of Spinal Stenosis and Methods for Using the Same

(76) Inventors: Richard S. Ginn, Gilroy, CA (US); David A. White, Morgan Hill, CA (US); John H. Ream, Willits, CA (US); Joseph Karratt, Santa Clara, CA (US); Tyler L. Baughman, San Jose, CA (US); Nicanor Domingo, Santa Clara, CA (US)

Correspondence Address: ORRICK, HERRINGTON & SUTCLIFFE, LLP;IP PROSECUTION DEPARTMENT, 4 PARK PLAZA, SUITE 1600, IRVINE, CA, 92614-2558, US

(21) Appl. No.: 12/352,796

(22) Filed: Jan. 13, 2009

Related U.S. Application Data
(60) Provisional application No. 61/045,169, filed on Apr. 15, 2008.
(60) Provisional application No. 61/144,070, filed on Jan. 12, 2009.

(51) Int. Cl. A61F 2/44

(52) U.S. Cl. 623/17.16

(57) ABSTRACT

Spacer devices for treating spinal stenosis are provided herein. In some example embodiments, these devices are configured for attachment on the interspinous ligaments with minimal injury thereto. Also provided are systems for the delivery of the spacer devices, tools for measuring and assessing the interspinous space and methods for using the same.

### CROSS-REFERENCE TO RELATED APPLICATIONS

The present application claims priority to U.S. provisional patent application Ser. No. 61/045,169, filed Apr. 15, 2008 and U.S. provisional patent application Ser. No. 61/144,070, filed Jan. 12, 2009, both of which are fully incorporated by reference herein.

### FIELD OF THE INVENTION

The subject matter described herein relates generally to the treatment of spinal stenosis and more particularly, to interspinous spacer devices and systems for the implantation of those devices and methods for using both.

### BACKGROUND OF THE INVENTION

Spinal stenosis is a condition in which a narrowing of the spinal canal leads to compression of the surrounding spinal tissue, which can include the spinal cord or spinal nerves. Spinal stenosis can be caused by a number of factors, but is most commonly attributed to the natural process of spinal degeneration that occurs with aging. It has also been attributed to causes such as spinal disc herniation, osteoporosis or the presence of a tumor.

Spinal stenosis can occur locally or globally anywhere along the spinal column. When limited to a local region, spinal stenosis is most commonly found in the lumbar region and, to a lesser extent, in the cervical region. Spinal stenosis can result in numerous symptoms that are generally dependent upon the location along the spine in which the stenosis occurs. For instance, cervical spinal stenosis can result in spastic gait, numbness or weakness in upper and/or lower extremities, radicular pain in the upper limbs as well as various other muscular, intestinal and/or nervous system abnormalities. Lumbar spinal stenosis typically results in lower back pain as well as pain or abnormal sensations in the legs, thighs or feet, as well as some intestinal and/or nervous system abnormalities.

Treatment for spinal stenosis generally seeks to create additional space for the affected nerves by removing surrounding tissue or bone and/or distracting the adjacent vertebral bodies, thereby relieving the nerve compression causing the patient's symptoms. Treatment can vary from complicated surgical procedures (e.g., laminectomy and/or foraminotomy in the lumbar region, and laminectomy, hemilaminectomy and/or decompression in the cervical region), to the rigid fixation of adjacent vertebral bodies in relation to each other (e.g., spinal fusion), to the implantation of interspinous spacer devices that distract affected vertebrae without rigid fixation.

Of these, the implantation of an interspinous spacer is generally the most preferred option for the patient since the surgical implantation procedure is relatively less invasive than spinal fusion and the patient retains more freedom in movement. Although the implantation of these devices is less invasive than spinal fusion or surgical tissue/bone removal, these devices still require the surrounding tissue to be dissected, modified and even resected to create adequate space in which the spacer can be implanted. For instance, the X-STOP device, offered by KYPHON (MEDTRONIC), requires a sizable incision through the interspinous ligament to allow the creation of a pocket in the ligament in which the device can be permanently implanted. This implantation procedure also requires the soft tissue adjacent the spinal column to be displaced and disrupted to provide enough room within the opened cavity for the physician to position, assemble and implant the device.

Another example is the COFLEX device, offered by PARADIGM SPINE LLC, which requires dissection of the supraspinous ligament to grant access to the interspinous space and then total resection of the interspinous ligament and any spinous process overgrowth to create a cavity in which the device can be implanted. This is further to the displacement and modification of surrounding soft tissue. Other interspinous spacer devices, such as the DIAM SPINAL STABILIZATION SYSTEM offered by MEDTRONIC SOFAMER DANEK,require similar or even more extensive injury to the spinal ligaments and surrounding tissues. Such invasive medical procedures can result in serious complications (e.g., nerve damage, infection, etc.) and discomfort for the patient.

Accordingly, improved interspinous spacer devices that can be implanted while avoiding the same degree of surgical disruption to the spinal ligaments and adjacent soft and hard tissue are needed.

### SUMMARY

Example embodiments of interspinous spacer devices, delivery systems, measurement tools and methods for using the same are described herein. Example embodiments of the interspinous spacer devices generally include two opposable arm-like members configured to reside on opposite sides of the interspinous tissue between adjacent vertebral bodies requiring distraction. Embodiments of the spacer can be implanted without causing significant injury to the spinal ligaments and surrounding tissue. Embodiments of the spacer device can include tissue-piercing anchors that, while traversing the interspinous ligament, cause only minimal injury to that tissue.

Other systems, methods, features and advantages of the invention will be or will become apparent to one with skill in the art upon examination of the description herein. It is intended that all such additional systems, methods, features and advantages be included within this description, be within the scope of the subject matter described herein, and be protected by the accompanying claims. In no way should the features of the example embodiments be construed as limiting the appended claims absent express recitation of those features in the claims.

### BRIEF DESCRIPTION OF THE FIGURES

The details of the invention, both as to its structure and operation, may be gleaned in part by study of the accompanying figures, in which like reference numerals refer to like parts. The components in the figures are not necessarily to scale, emphasis instead being placed upon illustrating the principles of the invention. Moreover, all illustrations are intended-to convey concepts, where relative sizes, shapes and other detailed attributes may be illustrated schematically rather than literally or precisely.

FIG. 1A is a perspective side view of a spinal column.

FIG. 1B is a side view of three lumbar vertebrae of a spinal column.

FIG. 1C is a top down view of a lumbar vertebral body.

FIG. 2A is a perspective view depicting an example embodiment of an interspinous spacer.

FIGS. 2B-C are cross-sectional, top down views depicting an example embodiment of an interspinous spacer.

FIG. 2D is a side view of a patient's spinal column depicting an example embodiment of an interspinous spacer thereon.

FIG. 2E is a planar cross-sectional view depicting an example embodiment of an interspinous spacer.

FIGS. 2F-G are perspective views depicting an example embodiment of an interspinous spacer.

FIGS. 2H-J are top-down views depicting an example embodiment of an interspinous spacer.

FIG. 3A is a perspective view depicting an example embodiment of an interspinous spacer.

FIGS. 3B-C are side views depicting an example embodiment of an interspinous spacer.

FIGS. 4A-C are perspective views depicting example embodiments of an interspinous spacer.

FIGS. 5A-6B are partial cross-sectional views of an example embodiment of a delivery system.

FIG. 7A is a perspective view depicting an example embodiment of an interspinous spacer.

FIG. 7B is an end on view depicting an example embodiment of an interspinous spacer and adjacent spinous processes.

FIG. 8A is a top down view depicting an example embodiment of an interspinous spacer.

FIG. 8B is a perspective view depicting an example embodiment of an interspinous spacer.

FIG. 9A is a top down view depicting an example embodiment of an interspinous spacer.

FIG. 9B is a perspective view depicting an example embodiment of an interspinous spacer.

FIG. 9C is a top down view depicting an example embodiment of an interspinous spacer.

FIG. 9D is a perspective view depicting an example embodiment of an interspinous spacer.

FIG. 9E is a top down view depicting an example embodiment of an interspinous spacer.

FIG. 10A is a perspective view depicting an example embodiment of an interspinous spacer.

FIG. 10B is a top down view depicting an example embodiment of an interspinous spacer.

FIGS. 10C-E are top down views of an example embodiment of an interspinous spacer in relation to interspinous tissue.

FIGS. 11A-B are side views depicting an example embodiment of an interspinous spacer.

FIG. 11C is a perspective view depicting an example embodiment of an interspinous spacer.

FIGS. 11D-E are top down views of an example embodiment of an interspinous spacer in relation to interspinous tissue.

FIGS. 12A-13 are internal side views depicting additional example embodiments of an interspinous spacer.

FIG. 14 is a perspective view depicting an example embodiment of an interspinous spacer.

FIG. 15A is a perspective view depicting an example embodiment of an interspinous spacer.

FIG. 15B is a top down view depicting an example embodiment of an inlerspinous spacer.

FIG. 15C is an end-on view depicting an example embodiment of an interspinous spacer.

FIG. 15D is an bottom up view depicting an example embodiment of an interspinous spacer.

FIG. 15E is a side view depicting an example embodiment of an interspinous spacer.

FIG. 15F is a top down view depicting an example embodiment of an interspinous spacer.

FIG. 15G is an end-on view depicting an example embodiment of an interspinous spacer.

FIG. 15H is a top down view depicting an example embodiment of an interspinous spacer.

FIG. 15I is an end-on view depicting an example embodiment of an interspinous spacer.

FIG. 16 is a block diagram depicting example methods of implanting example embodiments of an interspinous spacer.

FIG. 17A is a perspective view depicting an example embodiment of an interspinous spacer.

FIG. 17B is a perspective view depicting an example embodiment of a delivery system.

FIG. 17C is a perspective view depicting an example embodiment of a portion of a delivery system.

FIG. 17D is an exploded perspective view depicting an example embodiment of a portion of a delivery system.

FIG. 17E is a perspective view depicting an example embodiment of a delivery system.

FIG. 17F is an exploded perspective view depicting an example embodiment of a delivery system.

FIGS. 17G-H are cross-sectional views depicting example embodiments of an interspinous spacer and a delivery system.

FIG. 17I is a perspective view depicting example embodiments of an interspinous spacer and a portion of a delivery system.

FIGS. 17J-K are perspective views depicting example embodiments of an interspinous spacer.

FIG. 17L is a perspective view depicting example embodiments of an interspinous spacer and prong-like removal members.

FIGS. 17M-O are perspective views depicting an example embodiment of an interspinous spacer.

FIG. 17P is a perspective view of a patient's spinal column depicting an example embodiment of an interspinous spacer thereon.

FIGS. 18A-D are perspective views depicting example embodiments of an interspinous spacer.

FIGS. 18E-F are top down views depicting an example embodiment of an interspinous spacer.

FIGS. 18E-F are top down views depicting an example embodiment of an interspinous spacer.

FIG. 18G is a cross-sectional perspective view depicting the example embodiment of FIG. 18E taken along line 18G-18G.

FIG. 18H is a perspective view depicting example embodiments of a spacer and delivery system during an implantation procedure.

FIG. 18I is a perspective view depicting an example embodiment of a delivery system.

FIG. 18J is a cross-sectional view depicting the example embodiment of a delivery system of FIG. 18H taken along line 18J-18J.

FIG. 18K is a side view depicting an example embodiment of a spacer.

FIG. 18L is a cross-sectional view depicting one side of an example embodiment of a spacer in position against a vertebral body.

FIGS. 19A-B are perspective views depicting example embodiments of an interspinous spacer and a portion of a delivery system.

FIGS. 20A-B are perspective views depicting an example embodiment of an interspinous spacer.

FIGS. 21A-C are perspective views depicting example embodiments of measurement tools.

### DETAILED DESCRIPTION

The interspinous spacer devices described herein can be implanted over the interspinous tissue, such as the interspinous ligament and supraspinous ligament, and therefore can avoid the need to create a surgical opening for housing the main connective portion of that spacer device in the interspinous tissue. This reduces the complexity of the surgical implantation procedure and avoids the necessary injury to the surrounding tissue that accompanies use of conventional spacer devices. The interspinous spacer devices described herein can also be configured to accommodate the presence of the supraspinous ligament and engage, or clamp, with only the interspinous ligament, substantially avoiding irritation or trauma to the supraspinous ligament and also the anteriorly located ligamentum flavum.

Also described herein are systems for the delivery of interspinous spacer devices for use by the administering physician or medical professional. In addition, methods for the use of the spacer devices and delivery systems are provided. These devices, systems and methods will be described herein the context of treatment of spinal stenosis in the lumbar region of the spine, although, it should be noted that these devices, systems and methods can be used to treat spinal stenosis at any location along the spinal column.

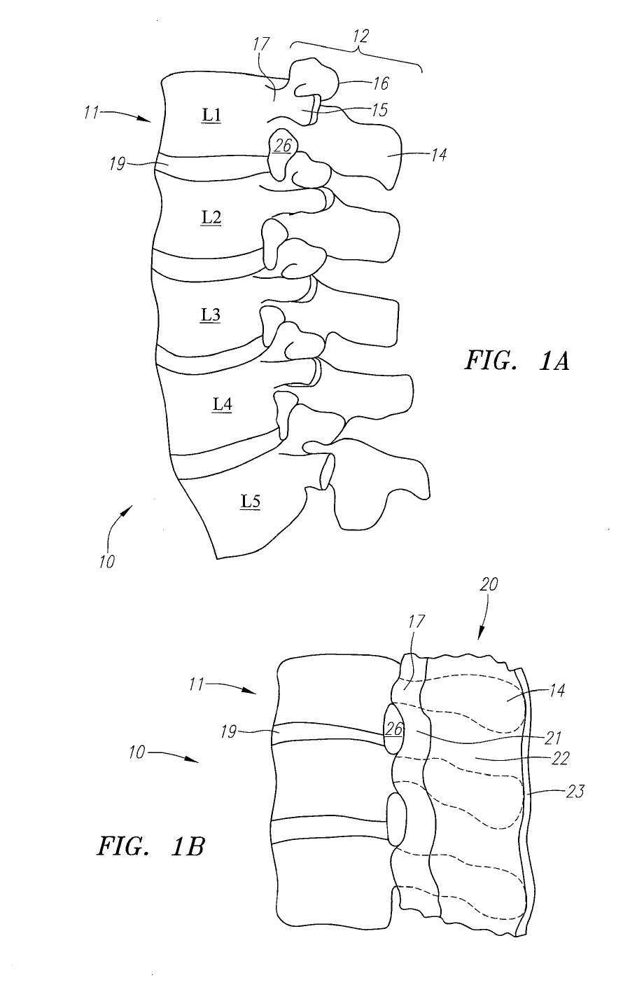

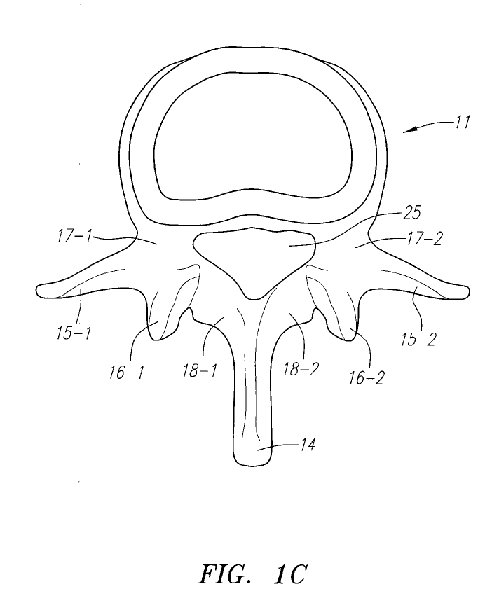

To better illustrate these devices, systems and methods, a description of the basic spinal anatomy will first be set forth. FIG. 1A is a perspective side view of a spinal column 10 showing five vertebral bodies 11, each separated by an intervertebral disc 19. More specifically, this region is the lumbar region of the spine and the five vertebral bodies 11 are lumbar vertebrae L1-L5. Each vertebral body 11 includes a posterior portion 12 having numerous bony features. The most prominent feature is spinous process 14, which is an elongate, fin-shaped feature that is situated the furthest posteriorly from each vertebral body 11. Located adjacent to spinous process 14 are left and right transverse processes 15 and left and right mamillary processes 16 (only the left side is shown here). These processes 14-16 are connected to each vertebral body 11 by way of left and right pedicles 17 (only left side shown).

FIG. 1B is a side view of three lumbar vertebrae of spinal column 10 with the left side pedicles 17 and processes 15-16 omitted to allow depiction of the interspinous tissue 20. Located adjacent to each vertebral body 11 and generally anterior to spinous process 14 (indicated as being obscured by dashed lines) is ligamentum flavum 21, which is immediately adjacent the intervertebral foramen 26. Posterior to ligamentum flavum 21, is the wider interspinous ligament 22 which extends alongside each spinous process 14. Posterior to interspinous ligament 22 is supraspinous ligament 23, which generally extends along the posterior edge of the interspinous tissue 20.

FIG. 1C is a top down view of a lumbar vertebral body 11. Here, left and right pedicles 17-1 and 17-2 can be seen in greater detail extending away from vertebral body 11. Also shown is spinous process 14, left and right transverse processes 15-1 and 15-2, mamillary processes 16-1 and 16-2 and left and right lamina 18-1 and 18-2. Between features 14-18 and the bulk of vertebral body 11 is a space referred to as the vertebral foramen 25. It is through the vertebral foramen 25 and intervertebral foramen 26 (shown in FIGS. 1A-B) that the spinal cord and other spinal nerves (not shown) are routed. Spinal stenosis is generally a narrowing or reduction in size of either or both of foramen 25-26 that results in the undesired compression of the nerves located therein.

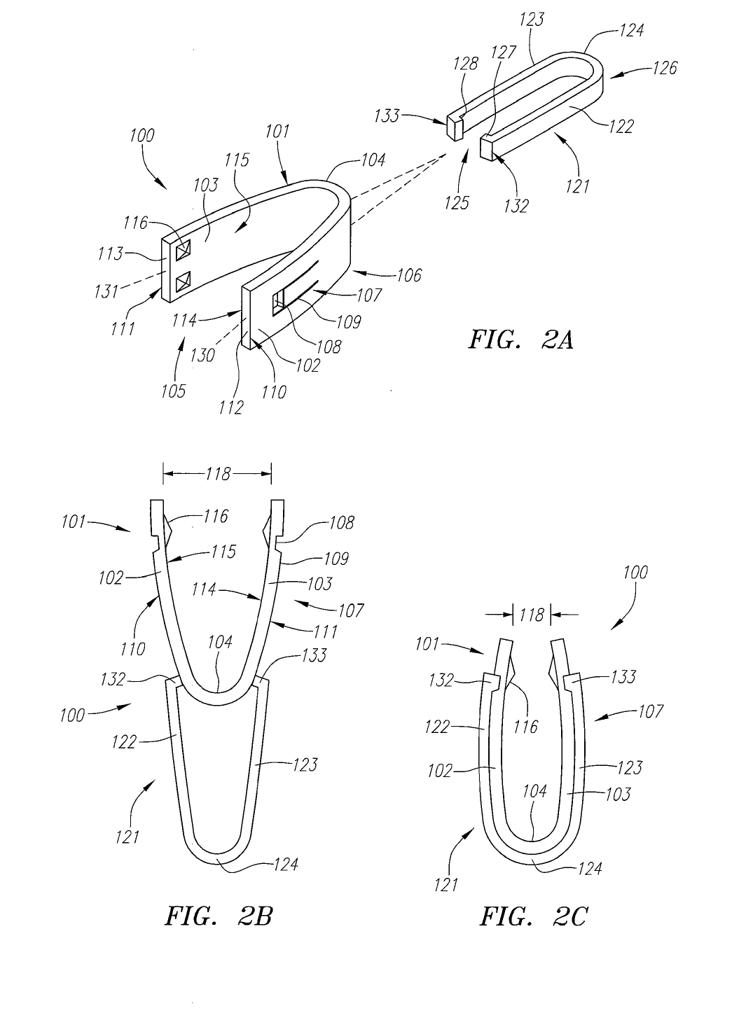

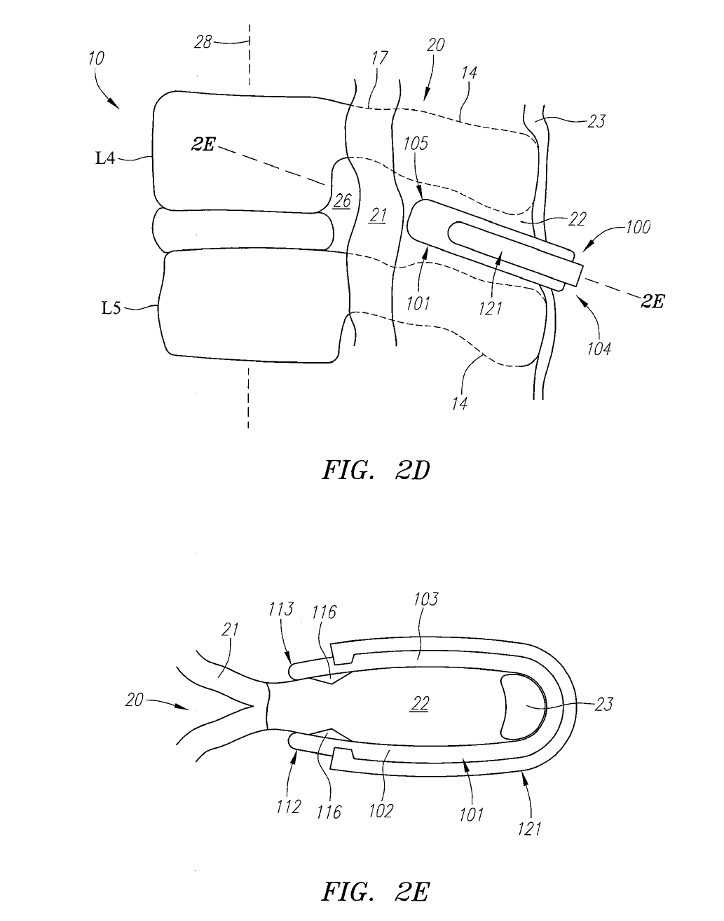

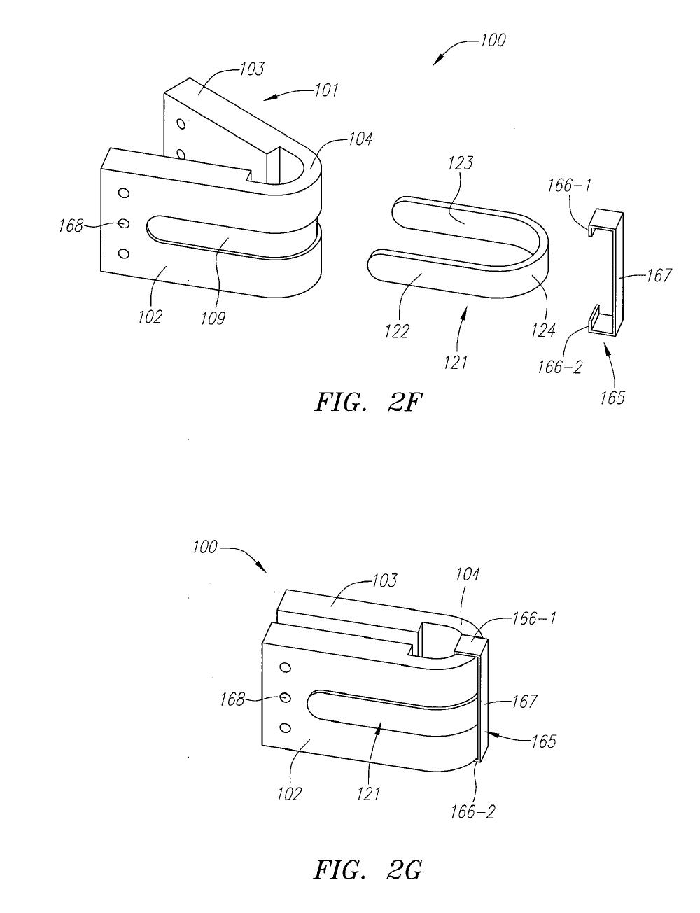

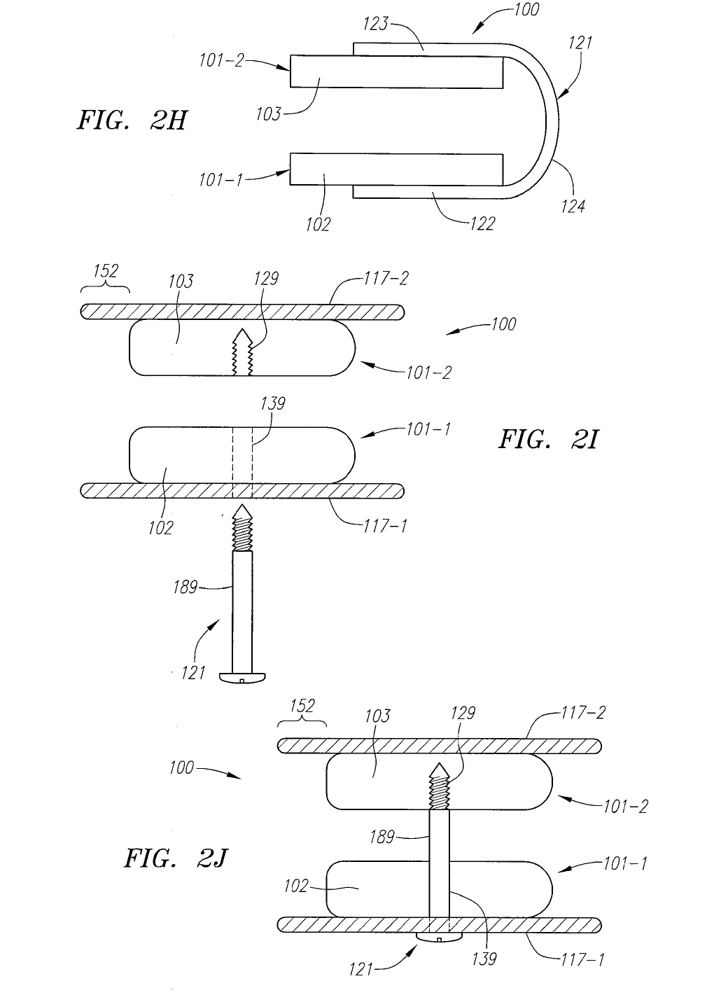

Turning now to the example embodiments, FIGS. 2A-E depict an example embodiment of a spacer device 100 configured for implantation within a patient. Here, spacer 100 is preferably configured for use as an interspinous spacer for placement over the interspinous and supraspinous ligament soft tissue present between adjacent spinous processes of the patient.

FIG. 2A is a perspective view depicting spacer 100 in an unassembled state. Here, spacer 100 includes two clip-like bodies, referred to herein as main spacer body 101 and closure body 121, which are configured to couple together. In this embodiment, main spacer body 101 includes a distal end 105 and a proximal end 106. A connective portion 104 is located at proximal end 106 and deflectably connects the proximal ends of a left spacing portion (or body) 102 and a right spacing portion 103 together, such that main spacer body is generally U-shaped. Any or all of left spacing portion 102, right spacing portion 103 and connective portion 104 can be configured to serve as the spacing portions of the device, i.e., acting as the load-bearing physical barrier to compressive movement between adjacent spinous processes. Any configuration that serves to space the adjacent spinous processes can be used. Here, left and right spacing portions are configured as being arm-like and will be referred to herein as arm portions. Portions 102 and 103 have a generally elongate, planar configuration but can be varied considerably, e.g., as will be described below. Distal ends 112-113 of arm portions 102-103, respectively, are preferably unconnected when in the open state depicted here.

Main spacer body arm portions 102-103 are preferably contractible, or capable of being drawn together, to transition from the open state to a relatively more closed state over the interspinous tissue, which will be described in more detail below. Similar to main spacer body 101, closure body 121 includes a connective portion 124 located at proximal end 126, which deflectably connects a left arm portion 122 and a right arm portion 123 together. Closure body arm portions 122-123 are preferably biased to deflect towards the closed state depicted in FIG. 2A.

FIG. 2B is a cross-sectional, top down view of spacer 100 as closure body 121 is in a preliminary state of advancement over main spacer body 101. The cross-section is taken along the plane intersecting central long axes 130 and 131 of arm portions 102 and 103, respectively (shown in FIG. 2A). Prior to implantation, main spacer body 101 is preferably in the relatively open state depicted here. Once positioned over the desired interspinous tissue within the patient's spinal column, closure body 121 can be slidably advanced over main spacer body 101 to close main spacer body 101 over the interspinous tissue. This can result in compression of the interspinous tissue. Closure of the device reduces the width 118 of the opening between arm portion distal ends 112-113.

FIG. 2C depicts main spacer body 101 in the closed state after closure body 1 21 has been advanced over main spacer body 101 and has been securely engaged thereto. As mentioned above, closure body 121 is preferably biased towards the closed state depicted in FIG. 2A. Main spacer body 101 can be biased towards the open state to facilitate the initial placement over the interspinous tissue, or can be configured without a pre-disposed bias, if desired. If main spacer body 101 is biased towards the open configuration, then closure body 121 is preferably capable of exerting sufficient compressive force to overcome the bias of main spacer body 101 and cause body 101 to close.

In the embodiment depicted in FIGS. 2A-C, each arm portion 102-103 preferably includes an interface feature 107 on the outer faces 110 and 111 of spacer body arm portions 102-103, respectively. Interface feature 107 is configured to interface with closure body 121 and can include a distally located indentation 108 and a proximally located guide track 109. Guide track 109 is preferably configured to guide the proper advancement of abutments 127 and 128, which are located on distal ends 132 and 133 of closure body left and right arm portions 122 and 123, respectively, into the respective indentations 108. Once in position, abutments 127-128 preferably snap into indentations 108 and provide feedback to the physician that closure body 121 has been fully seated over, and securely engaged with, main spacer body 101. It should be noted that other configurations can be used to couple bodies 101 and 121 together.

Main spacer body 101 can also include one or more tissue engaging features 116 on the tissue-contacting, inner faces 114 and 115 of left arm portion 102 and right arm portion 103, respectively. In this embodiment, tissue engaging feature 116 is a raised pyramidal portion of inner faces 114-115. In addition, or as an alternative, inner faces 114-115 can include grooves, dimples, a roughened surface or any other texturing that will increase surface friction with the adjacent tissue. Inner faces 114-115 can be configured such that, once implanted, they are either generally parallel or sloped with respect to each other or any combination thereof.

FIG. 2D is a side view of a patient's spinal column 10 depicting an example embodiment of spacer 100 implanted thereon. Vertebral bodies L4 and L5 are depicted with the pedicle 17, transverse process 15 and mamillary process 16 of the left side omitted to allow an unobstructed view of the interspinous tissue 20 and spacer 100. Spinous processes 14 as well as the right side pedicle 17 are shown obscured within this tissue by dashed lines.

Here, spacer 100 is shown clamped over interspinous ligament 22 and supraspinous ligament 23. Spacer 100 is preferably spaced a generally equal distance from the spinous processes 14 of L4 and L5. Spacer 100 is preferably configured such that distal end 105 does not extend past interspinous ligament 22 and does not contact ligamentum flavum 21. Spacer 100 can be configured to extend past interspinous ligament 22 and contact ligamentum flavum 21 if desired, however care should be taken to avoid contact with the spinal nerves (not shown) extending through intervertebral foramen 26 as well as the dura (also not shown).

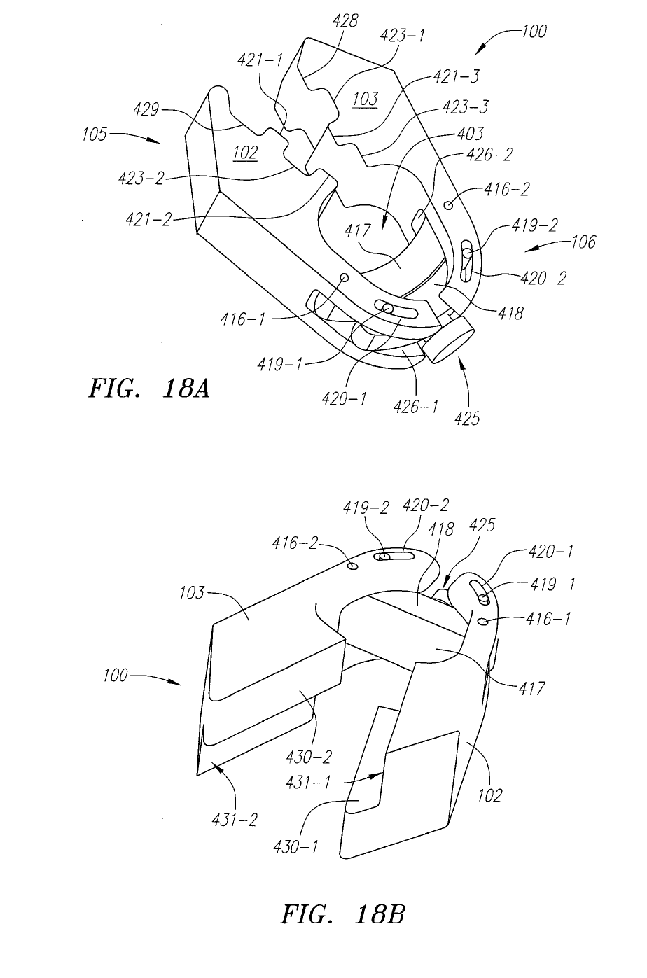

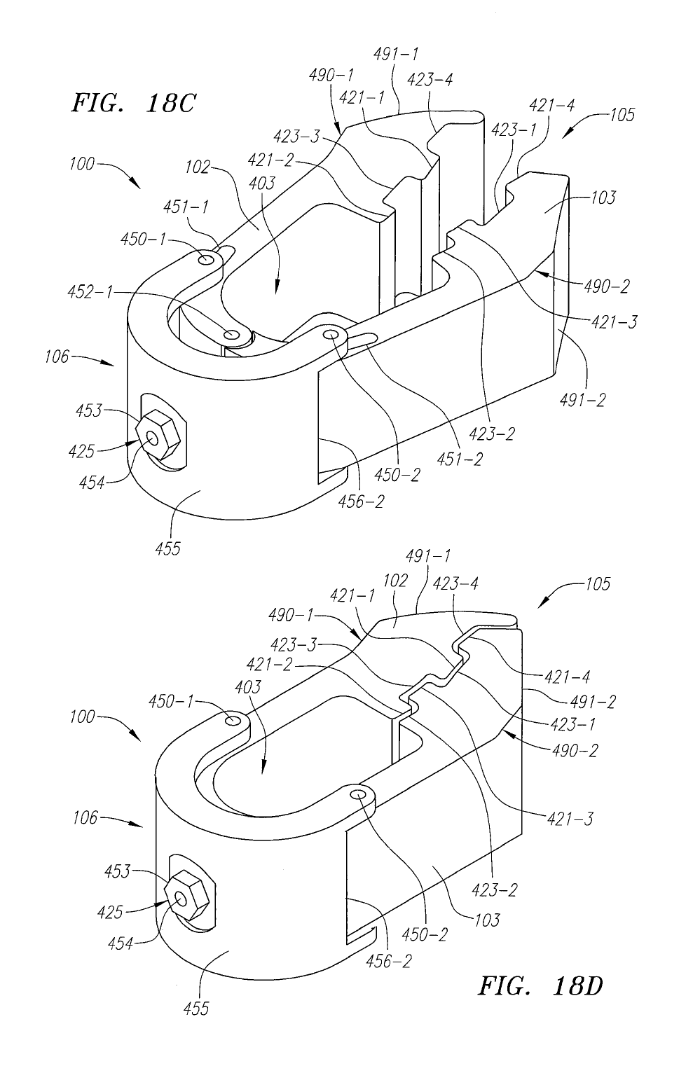

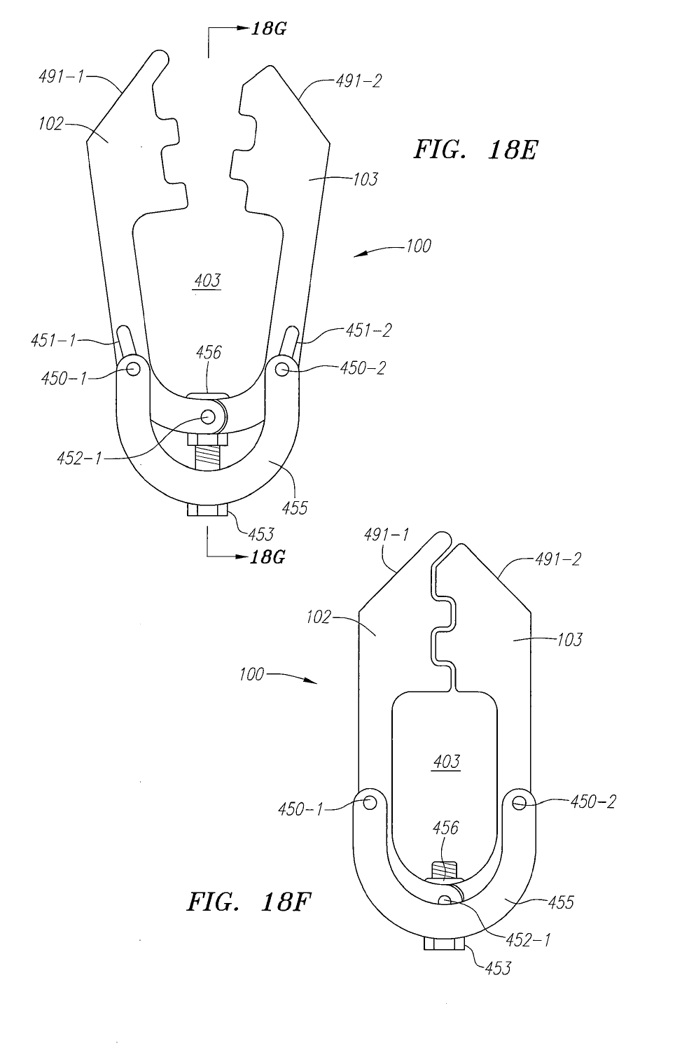

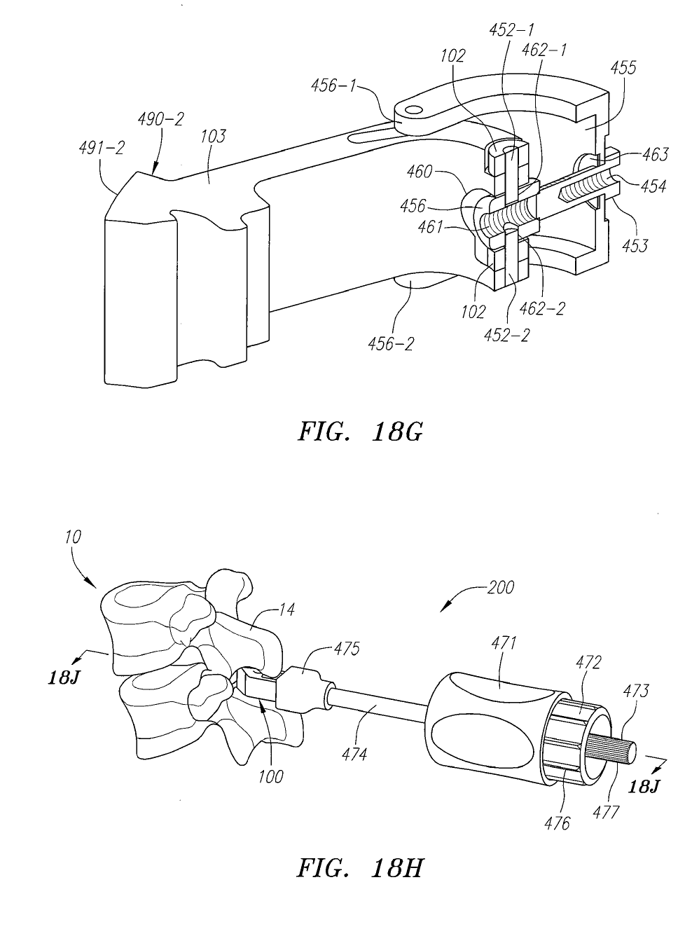

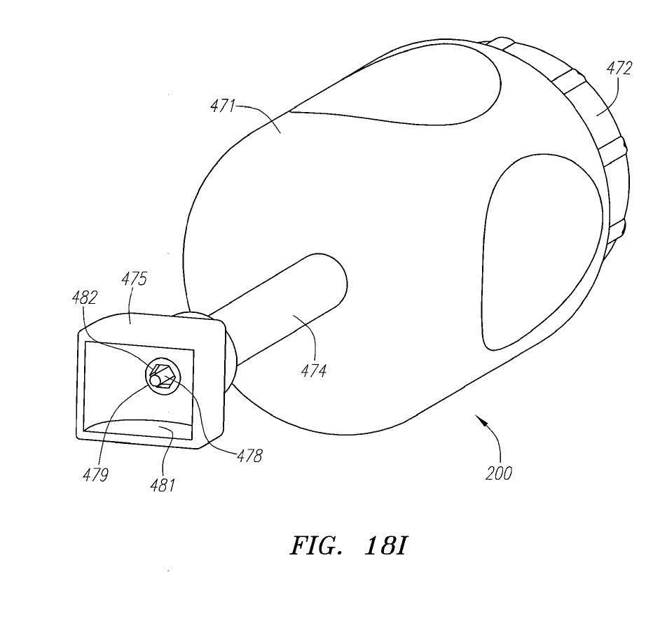

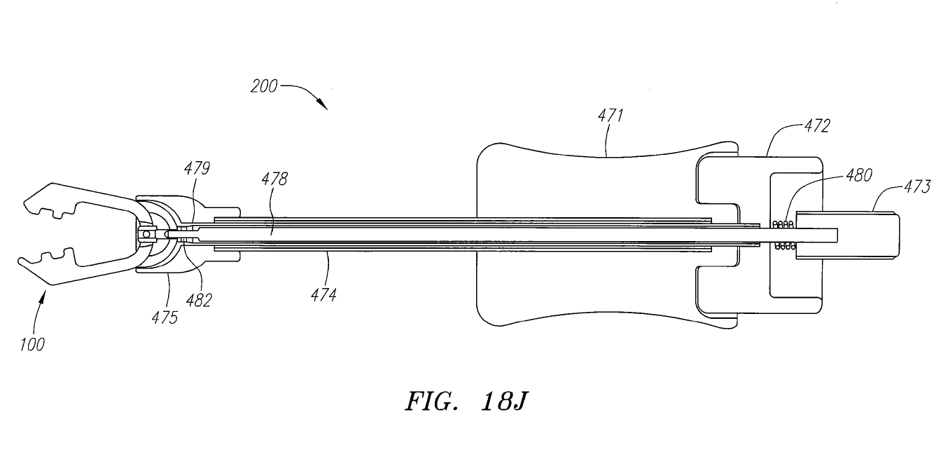

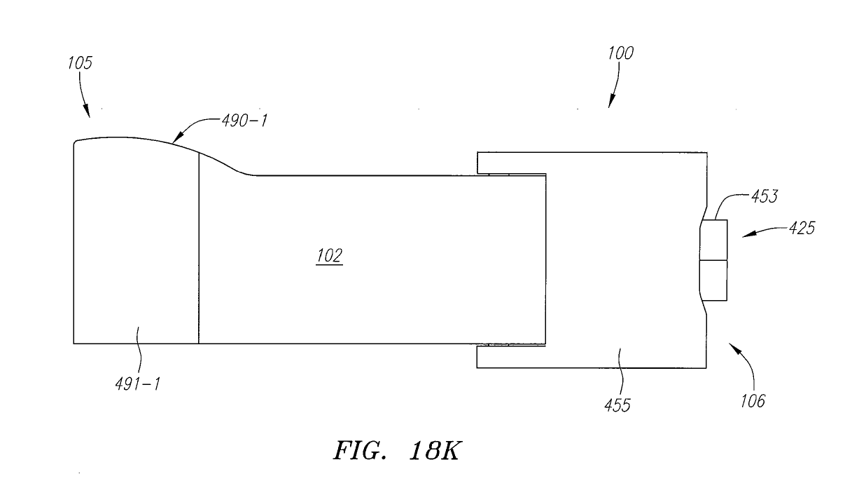

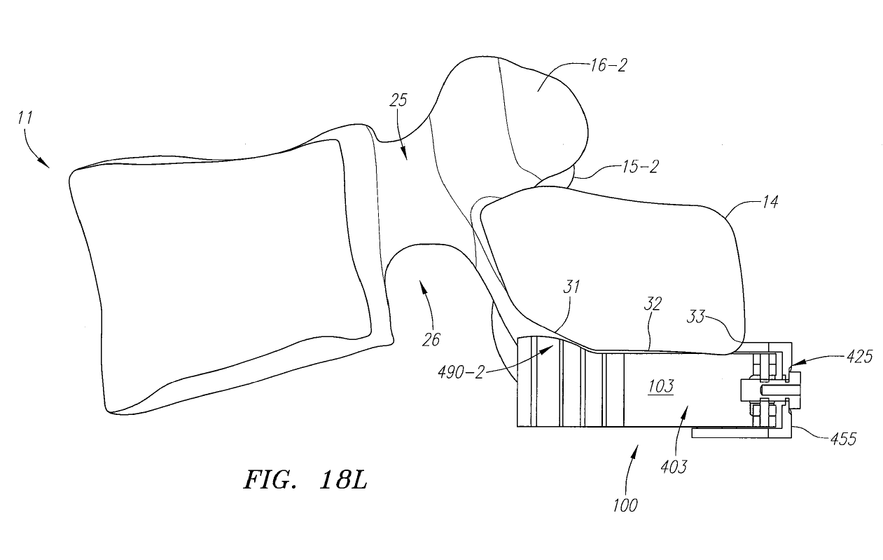

Here, it can be seen that spacer 100 is oriented at an angle with respect to the longitudinal axis 28 of spinal column 10. This is to account for the generally inferior inclination of spinous processes 14 as each extends posteriorly. It should be noted that although spacer 100 is depicted as being generally rectangular, spacer 100 can be shaped in any desired manner to account for the somewhat irregular profile of spinous processes 14. For instance, referring to the vertebral bodies L4 and L5 depicted here, distal end 105 can be made relatively taller so as to more completely engage the height of the interspinous ligament 22 present between L4 and L5 (see, e.g., embodiments described with respect to FIGS. 18K-L).

FIG. 2E is a planar cross-sectional view of an example embodiment of spacer 100 after implantation over the interspinous tissue taken along line 2E-2E of FIG. 2D. Here, it can be seen that spacer 100 is fit closely over interspinous ligament 22 and supraspinous ligament 23 with main spacer body distal ends 112 and 113 located adjacent to, but not contacting, ligamentum flavum 21. Spacer 100 preferably compresses the interspinous tissue by a degree sufficient that the tissue itself contributes significantly to distraction of the vertebral bodies and significantly resists compression past the desired stopping point of the adjacent processes 14. Spacer 100 can also be configured to compress the interspinous tissue to a degree where the one or more arm portions 102 and 103 reside directly between the adjacent spinous processes, thereby acting as a direct stop to compression.

Spacer 100 preferably exerts sufficient compressive force on the interspinous tissue to prevent any (or non-negligible) deflection of arm portions 102 and 103 towards the open state when the patient's normal movement forces the adjacent spinous processes together. To aid in this, connective portion 104 can be made relatively thicker than arm portions 102 and 103 for added strength. It should be noted that, although connective portion 104 is depicted as being a living hinge in FIGS. 2A-E and elsewhere, connective portion 104 can be configured in any other manner desired using one or more mechanical components.

It should be noted that conventional devices rely on the presence of a trans-interspinal tissue spacing portion, which is a relatively wide strut or other member placed through the interspinous tissue, or across a man-made interspinous space, to act as the load-bearing physical barrier to compressive movement between the adjacent spinous processes. The embodiments of spacer 100 described herein can stop the movement of the adjacent spinous processes without the use of such a trans-interspinal tissue spacing portion. Preferably, the height of left and right arm portions 102 and 103, when in close proximity with the adjacent spinous processes and in position on opposing sides of the interspinous tissue, act as the load-bearing structure providing the desired barrier to movement, or spacing distance, between the adjacent spinous processes.

It should be noted that any surface of spacer 100 that contacts tissue after implantation can be configured to be atraumatic to the surrounding tissue, including one or more of the spinous processes, by rounding the surfaces of the device, and/or by the use of coatings such as conformable coatings (e.g., sponge-like, gel-like) that are self-adapting to each patient's body anatomy. Also, the atraumatic coatings can be ones that inhibit inflammatory response (as will be described below) and the like.

Here, distal ends 112 and 11 3 are rounded to reduce irritation caused by friction with the surrounding tissue (e.g., the interspinous tissue between arm portions 102-103 and any tissue outside of arm portions 102-103).

Main spacer body 101 and closure body 121 (and any other component of spacer 100 described herein) can be formed from any number or types of materials that are suitable for the needs of the individual application. Each of bodies 101 and 121 can be formed from elastic (or superelastic) shape memory materials, i.e., materials that can exhibit a bias to revert towards a predetermined shape or state, such as nickel-titanium alloys (e.g., nitinol) and the like. This bias can be present before and after implantation or can be configured to initiate once a predetermined temperature is reached (e.g., slightly below human body temperature). Other suitable materials include titanium, stainless steel, cobalt chrome (e.g., elgiloy) and various polymers such as polyetheretherketones (PEEK) and the like. Materials that are not magnetic can allow compatibility with magnetic resonance imaging (MRI) systems. Materials that approximate bone density, such as PEEK, can minimize trauma to the adjacent spinous processes. Each of bodies 101 and 121 can also be formed from the same or different materials. Spacer 100, as well as any portion thereof, can be formed from only one body (monolithic) or more than one body (multiple discrete bodies). For example, arm portions 102 and 103 can be discrete components formed from a rigid (i.e., inflexible) material and connective portion 104 can be a component formed from a more flexible material, for instance, to ease bending in that regions or to minimize irritation to the supraspinous ligament. Furthermore, any portion of spacer 100 can be coated with any desired material, such as bio-compatible substances, substances to alter the surface friction (either increase or decrease) between the device and any surrounding tissue, substances to promote healing, atraumatic and conformable substances as described earlier, absorbable and other substances to promote the growth of scar tissue or other tissue (e.g., poly-L-lactide (PLLA), polyglycolide (PGA), sheep intestinal submucosa, etc.), and the like.

FIGS. 2F-G are perspective views depicting another example embodiment of spacer 100 in unassembled and assembled states, respectively. Here, spacer 100 includes a closure body retainer 165 configured to securely couple with main spacer body 101 over top of closure body 1 21 to retain the closure body 121 on main spacer body 101. Here, retainer 165 includes clips 166-1 and 166-2 on the upper and lower sides of the elongate body 167. Retainer 165 is preferably formed from a material with sufficient elasticity to allow the clips 166 to be deflected and positioned over main spacer body 101. Retainer 165 is shown locked in place over the connective portion 104 of main spacer body 101, although retainer 165 can be placed in any location that allows retention of closure body 121. It should be noted that retainer 165 is not limited to the use of clips as any fastening or locking structure can be used instead.

In this embodiment, guide track 109 is a recess shaped complementary to closure body 121 and is present over the entire region of main spacer body 101 where closure body 121 is intended for placement. Guide track 109 is preferably configured such that closure body 121 sits flush against the main spacer body 101. This provides a lower overall profile and can reduce any irritation or inflammation that may result from tissue contact with an uneven spacer surface. The use of retainer 165 can allow for the omission of any features for interlocking closure body 121 with main spacer body 101 (e.g., abutments 132 and 133 and indentations 108).

Although retainer 165 and closure body 121 are shown here as separate elements, it should be noted that these elements can be combined into a single closure body that both closes main spacer body 101 and clips or otherwise fastens to main spacer body 101.

Also, left and right arm portions 102 and 103 are relatively thicker than connective portion 104 in this embodiment. Variation of the thickness in this manner can allow sufficient rigidity for arm portions 102 and 103 while allowing greater flexibility and/or reduced stress along connective portion 104.

It should also be noted that, in certain cases, it may be desirable to fasten spacer 100 in place over the interspinous tissue with the use of fasteners or sutures. Here, left and right arm portions 102 and 103 include apertures 168, through which the physician can pass a needle and suture to fasten spacer 100 in place. The number and placement of apertures 168 can be varied as desired.

FIG. 2H is a top down view depicting another example embodiment of spacer 100. Here, spacer 100 includes two separate main spacer bodies 101-1 and 101-2, which are configured as left and right spacing portions 102 and 103, respectively. Connective portion 104 has been omitted. To implant this embodiment, the physician can position each of portions 102 and 103 separately, and then couple the portions together using a U-shaped closure body 121 or other attachment mechanism.

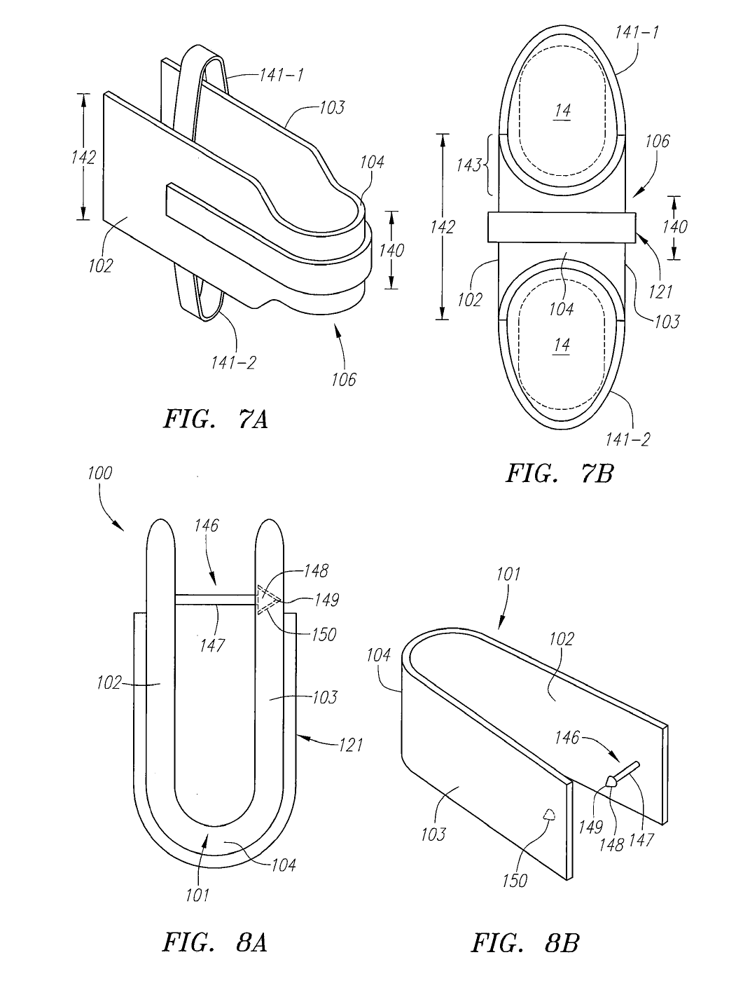

FIGS. 2I-J are top down views depicting another example embodiment of spacer 100 having separate main spacer bodies 101-1 and 101-2. Here, each of spacer bodies 101-1 and 101-2 are again configured as left and right spacing portions 102 and 103 and one or both can include plate-like outer members 117-1 and 117-2. Plate-like outer members 117 have portions 152, which extend past the inner bodies of portions 102 and 103, that are configured to be seated alongside the spinous processes located inferior and superior to the spacer 100, in a manner similar to wing portion 143, which are shown and described with respect to FIGS. 7A-B. Plate-like outer members 117 can be coupled to the inner members in any desired manner, such as by the use of welds, solder, adhesives, screws, clips, clamps and the like. Also, outer members 117 can be integrated with the inner member such that each body 101 has a monolithic (uni-body) construction.

In this embodiment, a screw-like attachment mechanism 189 is used to couple bodies 102 and 103 together. Screw 189 can have a tissue-piercing end, or can have a dull or atraumatic end and configured to be advanced through a pre-existing, man-made opening. Right arm portion 103 includes a threaded lumen 129 and left arm portion 102 includes a generally cylindrical lumen 139, both generally centrally located on bodies 101-1 and 101-2 and configured to receive screw 189.

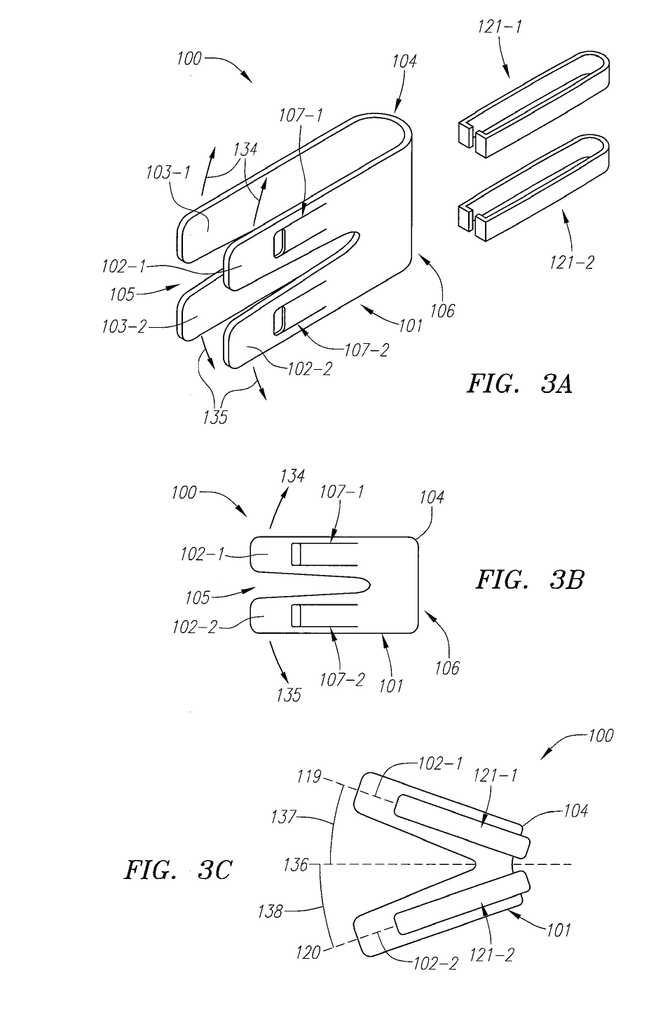

FIGS. 3A-C depict another example embodiment of spacer 100. FIG. 3A is a perspective view showing the components of spacer 100 in the unassembled configuration. Here, main spacer body 101 includes two left arm portions 102-1 and 102-2 as well as two corresponding right arm portions 103-1 and 103-2. Each arm portion can also include an interface feature 107. Two closure bodies 121-1 and 121-2 are also shown, each being configured to slidably couple with main spacer body 101 and to close the respective arm portions 102-103.

In this embodiment, in addition to clamping onto the interspinous tissue, main spacer body 101 has an adjustable height and is biased to expand in superior and inferior directions towards the adjacent spinous processes. More specifically, superiorly located arm portions 102-1 and 103-1 are biased to deflect in superior direction 134 and inferiorly located arm portions 102-2 and 103-2 are configured to deflect in inferior direction 135. FIG. 3B and FIG. 3C are side views of spacer 100 before and after deflection, respectively. By deflecting to the heightened or expanded state of FIG. 3C, spacer 100 can be self-adjusting to the needed spacing height between adjacent spinous processes.

One of skill in the art will readily recognize that a stronger bias to expand the height of main spacer body 100 will result in a relatively greater force applied against the adjacent spinous processes. This bias can be tailored according to the needs of the individual patient.

The degree of deflection in directions 134 and 135 can vary as desired for the particular application. Preferably, superiorly located portions 102-1 and 103-1 deflect in direction 134 by an angle 137 between zero and 60 degrees, as measured between superior arm portion longitudinal axis 119 and central longitudinal axis 136. Likewise, inferiorly located portions 102-2 and 103-2 deflect in direction 135 by an angle 138 between zero and 60 degrees, as measured between inferior arm portion longitudinal axis 120 and central longitudinal axis 136. As depicted here, deflection angles 137 and 138 are both approximately 30 degrees. It should be noted that deflection angles 137 and 138 can be configured to deflect by similar or different amounts. In addition, the pair of left and right arm portions 102 and 103, located either superiorly or inferiorly, can each be configured to deflect by different amounts if desired.

It should be noted that although two closure bodies 121-1 and 121-2 are described in this embodiment, a single closure body 121 having a shape corresponding to that of main spacer body 101 can also be used, the single closure body 121 also being manually deflectable or biased to deflect in an manner similar to body 101. In an alternative embodiment, a single coupling device 121 configured to interface with each of arm portions 102-103, can be biased to close each pair of arm portions 102-103 as well as to deflect each pair of arm portions 102-103 in the appropriate, superior or inferior, direction. In such an embodiment, main spacer body 101 can be deflectable but can be configured without any predisposed biased to deflect, said bias being provided by the single closure body 121.

It should be noted that spacer 100 can be delivered with any desired sequence of closure over the interspinous tissue and height expansion. For instance,.spacer 100 can first be closed over the interspinous tissue and then expanded superiorly and inferiorly or, vice-versa. In one example embodiment, main spacer body 101 is composed of a thermally-actuatable shape memory material such as nitinol. Main spacer body 101 can first be positioned and closed over the interspinous tissue by the physician. Main spacer body can be configured to expand superiorly and/or inferiorly after implantation, by activating the shape memory characteristics of body 101 once the temperature of the patient's body reaches a predetermined level, preferably set near the normal human body temperature.

While many embodiments of spacer 100 are described herein as incorporating at least one closure body 121 to aid in closure, it should be noted that closure body 121 is not required. For instance, spacer 100 can be closed manually and then locked in the closed state or spacer 100 can be self-closable. For instance, if main spacer body 101 is configured to be self-closable, left and right arm portions 102 and 103 are preferably biased to deflect towards each other and into the closed state without the aid of a supplemental closure body 121. In a self-closable embodiment, main spacer body 101 is preferably composed of nitinol (or other shape memory material) and is heat treated in the desired closed state to instill a bias in portions 102 and 103 to revert towards that closed state whenever deflected into another position (e.g., the open state). In another embodiment, left and right portions 102 and 103 can be magnetized or can include magnetic portions that aid in closure.

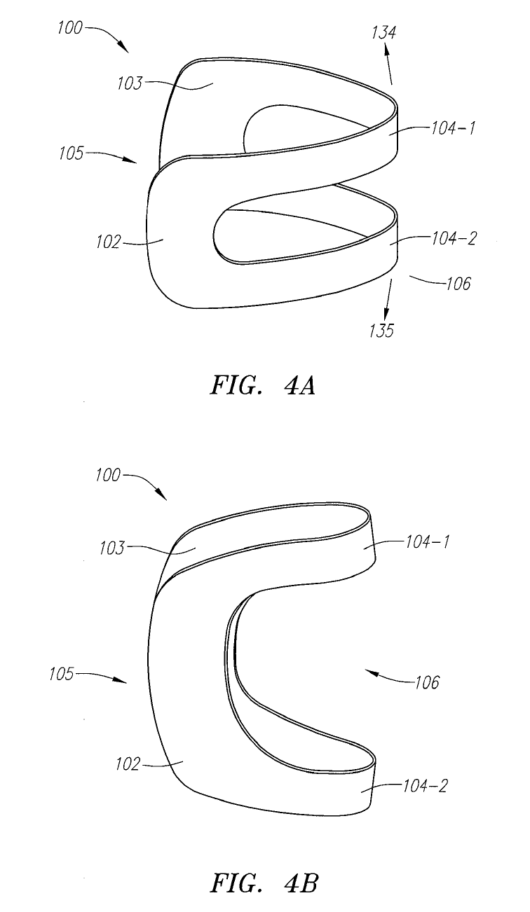

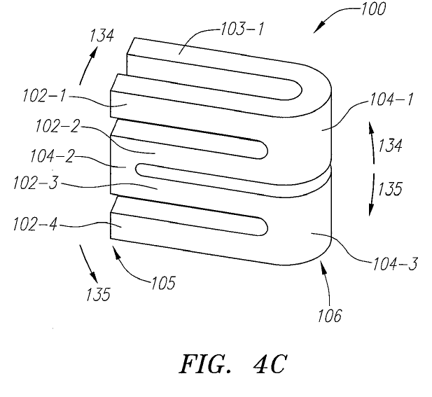

FIGS. 4A-B are perspective views depicting an example embodiment of spacer 100 that is self-closable as well as biased to expand in superior direction 134 and inferior direction 135. Here, spacer 100 is preferably composed of nitinol and biased to transition from the open, unexpanded state of FIG. 4A towards the closed, expanded state of FIG. 4B. Spacer 100 includes left arm portion 102 and right arm portion 103 at distal end 105, which transition to upper and lower connective portions 104-1 and 104-2, respectively, at proximal end 106. Spacer 100 is biased such that connective portions 104-1 and 104-2 separate to expand the height of spacer 100, similar to the embodiment described with respect to FIGS. 3A-C.

FIG. 4C is a perspective view depicting another example embodiment of spacer 100 biased to expand. Here, spacer 100 is shown in the unexpanded state and both distal end 105 and proximal end 106 of spacer 100 are expandable in directions 134 and 135, respectively. Spacer 100 includes a superior-most left arm portion 102-1 coupled with an immediately inferiorly located arm portion 102-2 via connective portion 104-1. Arm portion 102-2, in turn, is connected to an immediately inferiorly located arm portion 102-3 via connective portion 104-2 and the inferior-most arm portion 102-4 is connected to arm portion 102-3 via connective portion 104-3. Corresponding portions are also located on the right side of the device but only the left side is shown in detail here. By configuring both distal and proximal ends 105 and 106, respectively, to expand, spacer 100 is capable of applying a generally more uniform force upon the superiorly and inferiorly located bone and tissue. Any number of stacked arm portions can be used. It should be noted that the embodiments described with respect to FIGS. 3A-4C can find beneficial use in conjunction with regular discectomy or micro-discectomy procedures.

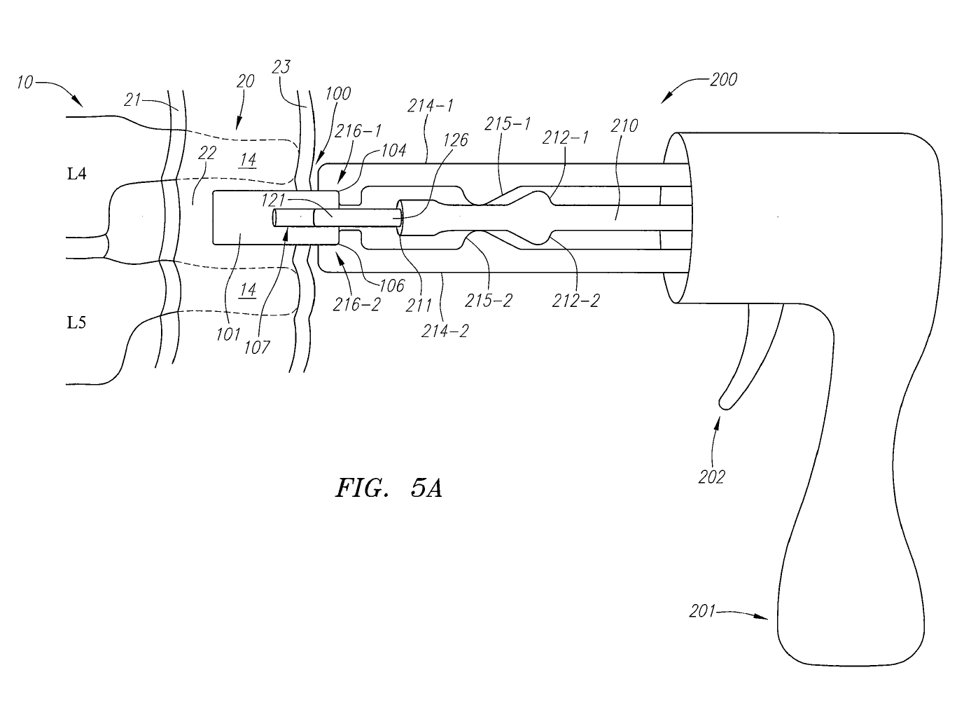

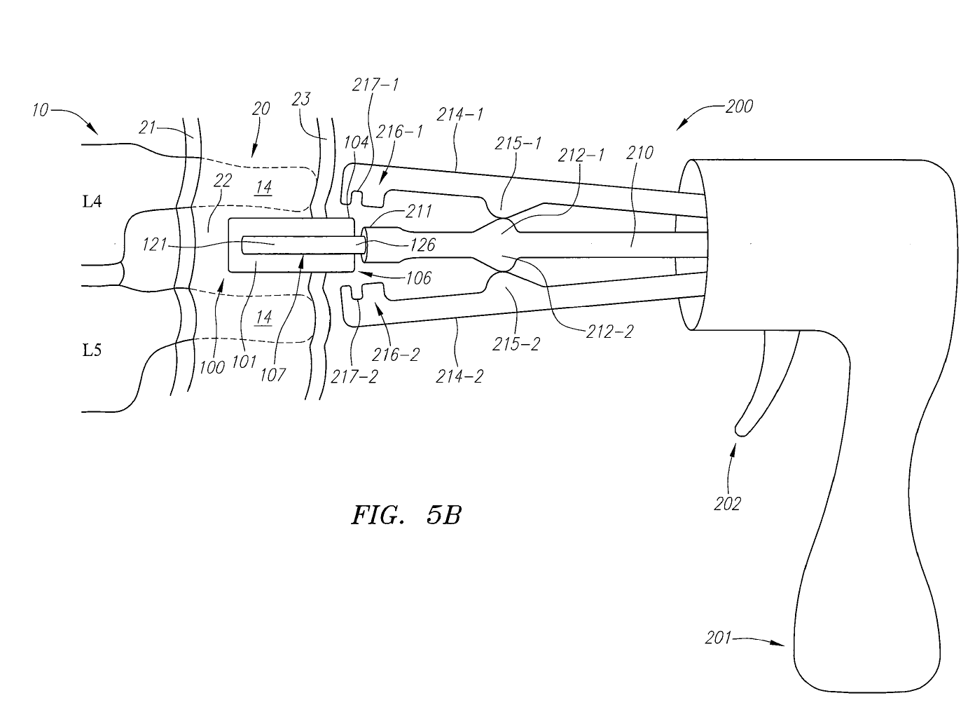

FIG. 5A is a partial cross-sectional view of an example embodiment of a manually actuated delivery system 200 for delivering spacer 100 to the desired location on the patient's spinal column 10 during an implantation procedure. Only the relevant portions of the patient's spinal column 10 are shown in a similar manner as with FIGS. 1B and 2D to facilitate illustration of the implantation and delivery of spacer 100. It should be noted that although this embodiment is shown and described coupled with a manually actuatable proximal portion, this embodiment can be configured for use with automatically or robotically actuated proximal portions as well.

Here, delivery system 200 includes a proximal handle portion 201, an actuator 202 (which in this embodiment is configured as a trigger), a pusher member 210 and opposed expandable engagement members 214-1 and 214-2 (which are configured to engage and hold proximal end 106 of spacer 100 during delivery). Although not shown, the proximal ends of expandable engagement members 214 are pivotably coupled with proximal handle portion 201 to allow the members 214 to expand or pivot apart when release of spacer 100 is desired. Distal end 211 of pusher member 210 is preferably configured to engage proximal end 126 of closure body 121 with a recessed portion or other suitable interlocking interface. Pusher member 210 can be movable distally upon actuation by actuator 202. Pusher member 210 also includes abutments 212-1 and 212-2, which, in this configuration, are shown proximally located to corresponding abutments 215-1 and 215-2 on engagement members 214-1 and 214-2, respectively.

FIG. 5B is a similar view depicting delivery system 200 after actuation of actuator 202. Actuation of actuator 202 has caused pusher member 210 to be translated distally and has pushed closure body 121 over main spacer body 101, closing spacer 100 over the interspinous tissue 20. As pusher member 210 traveled distally, abutments 212-1 and 212-2 came into contact with the corresponding abutments 215-1 and 215-2 on engagement members 214-1 and 214-2, respectively. Continued advancement caused the sloped surfaces of abutments 212 and 215 to travel in contact with each other and force engagement members 214-1 and 214-2 apart, thereby releasing the grasp of delivery system 200 on spacer 100. It should be noted that one of skill in the art will readily recognize the many various mechanical and/or electrical interfaces that can be used to cause engagement members 214 to separate upon advancement of pusher 210. While in this embodiment, delivery system 200 is configured such that the expansion of members 214 is dependent on and occurs simultaneously with the advancement of pusher 210, it should be noted that these actions can occur independent of each other. In such an embodiment, actuator 202 can be configured to initiate each separate action or separate actuators can be included.

Distal ends 216 of engagement members 214 can be configured in any manner to engage and hold proximal end 106 of main body portion 101. Here, distal ends 216 each include a groove 217 configured to slide over connective portion 104 and securely engage main spacer body 101. Once released as depicted in FIG. 5B, delivery system 200 can be withdrawn and the surgical access opening within patient's back can be closed.

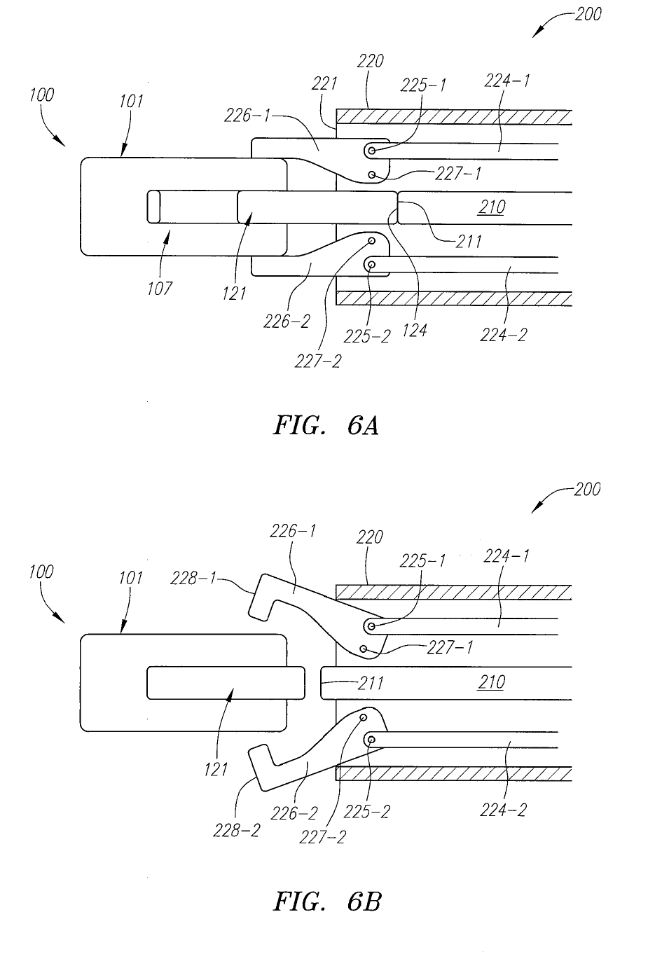

FIGS. 6A-B are partial cross-sectional views of the distal portion of another example embodiment of delivery system 200. This distal portion can be coupled with a proximal handle portion having an actuator, similar to the embodiment described with respect to FIGS. 5A-B. Here, delivery system 200 includes pusher member 210 in addition to two elongate struts 224-1 and 224-2, the distal ends of which are pivotably coupled with engagement members 226-1 and 226-2, which are in turn configured to engage and securely hold main body portion 101 of spacer 100. Engagement members 226 are in turn pivotably coupled with housing 220, which is depicted here in cross-section so as not to obstruct the view of the components located therein. Engagement members 226-1 and 226-2 are pivotably coupled with housing 220 by way of hinges 227-1 and 227-2. Likewise, elongate struts 224-1 and 224-2 are each pivotably coupled with engagement members 226-1 and 226-2 by hinges 225-1 and 225-2, respectively.

FIG. 6A depicts delivery system prior to or during positioning of spacer 100, while FIG. 6B depicts delivery system 100 after release of spacer 100. After advancement of pusher 210 (or simultaneous with advancement of pusher 210) to slide closure body 121 into position over main spacer body 101, elongate struts 224 can be proximally retracted to cause engagement members 226 to pivot about hinges 225 and 227. This causes engagement members 226-1 and 226-2 to swivel in clockwise and counterclockwise directions, respectively, thereby releasing the grasp of engagement members 226 on main body portion 101 and freeing spacer 100 from delivery system 200. After release, delivery system 200 can be withdrawn and the access opening to the patient's spinal column can be closed.

The embodiment of delivery system 200 depicted in FIGS. 6A-B requires a relatively smaller access opening due to the use of the pivotable engagement members 226 located on the distal end thereof. This can be advantageous because the access cavity does not need to be as large thereby reducing the risk of infection or other secondary complications to the patient.

Referring back to the various embodiments of interspinous spacers, FIGS. 7A-B depict another example embodiment of spacer 100. FIG. 7A is a perspective view and FIG. 7B is an end on view of proximal end 106 of spacer 100 after implantation next to spinous processes 14. Here, the height 140 of connective portion 104 is relatively less than the height 142 of arm portions 102 and 103. Arm portions 102 and 103 extend past connective portion 104 such that they extend superiorly and inferiorly to connective portion 104. These extended portions will be referred to herein as wing portions 143.

Wing portions 143 are preferably configured to extend alongside spinous processes 14 in such a manner that prevents rotation of clip spacer 100 within the interspinous region. In this embodiment, spacer 100 is also configured with a superiorly and inferiorly located tethers 141-1 and 141-2. These tethers are optional, but if included can be routed through or over the corresponding spinous process 14 to affix spacer 100 to the bone of the patient's spinal column. This can provide an advantage in more securely engaging the patient's spinal column for prevention of movement over the long term period of implantation. Tethers 141 can be composed of any flexible, bio-compatible material, such as DACRON and the like.

FIGS. 8A-B depict another example embodiment of spacer 100. FIG. 8A is a top down view of assembled spacer 100 in the closed state, while FIG. 8B is a perspective view of main body portion 101 in the open state. This embodiment of spacer 100 is configured with a distally located tissue anchor 146 configured to anchor spacer 100 with respect to the interspinous tissue. Tissue anchor 146 is preferably an elongate, needle-like tissue-piercing member that includes an elongate shaft 147 and an enlarged end portion 148 with a substantially sharp distal tip 149 configured to pierce the interspinous tissue. Tissue anchor 146 can be fixed to either of the left or right arm portions 102-103. Here, tissue anchor 146 is fixed to left arm portion 102, Right arm portion 103 preferably includes a recess 150 configured to receive and (optionally) securely engage enlarged head 148. It should be noted that because enlarged head 148 and recess 150 in FIG. 8A and recess 150 in FIG. 8B are obscured by main body portion 101, these elements are depicted with dashed lines to illustrate their positioning.

In this embodiment, main body portion 101 is preferably positioned over the location of implantation and closed on the interspinous tissue with force sufficient to cause tissue anchor 146 to pierce and travel through the interspinous tissue (e.g., the interspinous ligament) and into recess 150 where it can securely engage, or snap, into place. Once snapped into place, main spacer body 101 is locked in position on the spinal column 10. Once locked together, closure body 121 can be removed if desired. In an alternate embodiment, closure body 121 can be omitted altogether and the delivery system (or the physician) can apply the compressive force necessary to cause arm members 102 and 103 to deflect towards each other and lock in place, thereby alleviating the need for closure body 121 altogether. Although only one tissue anchor is shown in this embodiment, any number of anchors can be used. If multiple anchors are used, the anchors can be arranged in alternating fashion, such that each arm portion 102 and 103 has at least one tissue anchor fixed thereto.

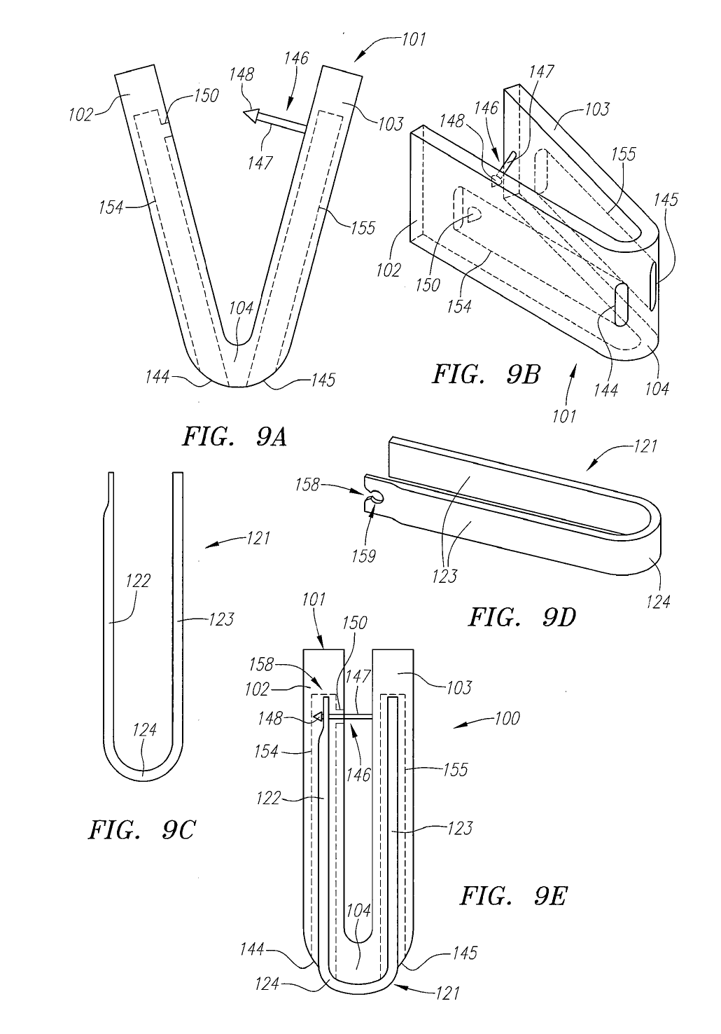

FIGS. 9A-E depict another example embodiment of spacer 100 and its various components. FIGS. 9A-B depict main body portion 101 with top down and perspective views, respectively, FIGS. 9C-D depict closure body 121 with top down and perspective views, respectively, and FIG. 9E is a top down view of spacer 100 in a closed assembled state. Elements or features that are obscured are shown to reflect this by the use of dashed lines. In this embodiment, main spacer body 101 has inner lumens 154 and 155 configured to slidingly receive closure body left arm portion 122 and right arm portion 123 through open proximal ends 144-145, respectively. This embodiment further includes tissue anchor 146, fixed to right arm portion 103, and recess 150, which opens into lumen 154 and is configured to receive tissue anchor enlarged portion 148. Lumens 154 and 155 are preferably shaped complementary to that of the profile of closure body 121. For instance, in this embodiment closure body 121 has a generally rectangular keyed cross-sectional profile, as does lumens 154 and 155.

FIGS. 9C-D depict closure body 121 in more detail. Here, closure body left arm portion 122 has a tapered distal end 158 that includes an interlocking feature 159 configured to securely engage, e.g., slidably snap over, tissue anchor shaft 147. After main body portion 101 has been positioned and closed over the interspinous tissue with tissue anchor 146 received within recess 150, closure body 121 is then preferably advanced along inner lumens 154 and 155. Continued advancement of closure body 121 distally into main body portion 101 allows tapered distal end 158 to slide over and onto tissue anchor shaft 147 into such a position where shaft 147 is securely retained by distal end 1 58.

In this embodiment, the force necessary to close main body portion 101 is preferably applied by the delivery system prior to insertion of closure body 121. In this case, closure body 121 does not have to exert any closure force while within lumens 154 and 155, although some closure force is desirable. In an alternative embodiment, the closure of main body portion 101 can be accomplished by the actual advancement of closure body 121 into lumens 154 and 155, where continued advancement draws the left and right arm portions 102-103 together. Main spacer body 101 preferably enters the closed state with elongate shaft 147 received within recess 150 and lumen 154 prior to the arrival of distal end 158, in order to allow distal end 158 to securely engage shaft 147. It should be noted that the interlocking distal end 158 of closure body 121 and tissue anchor 146 can be omitted if desired.

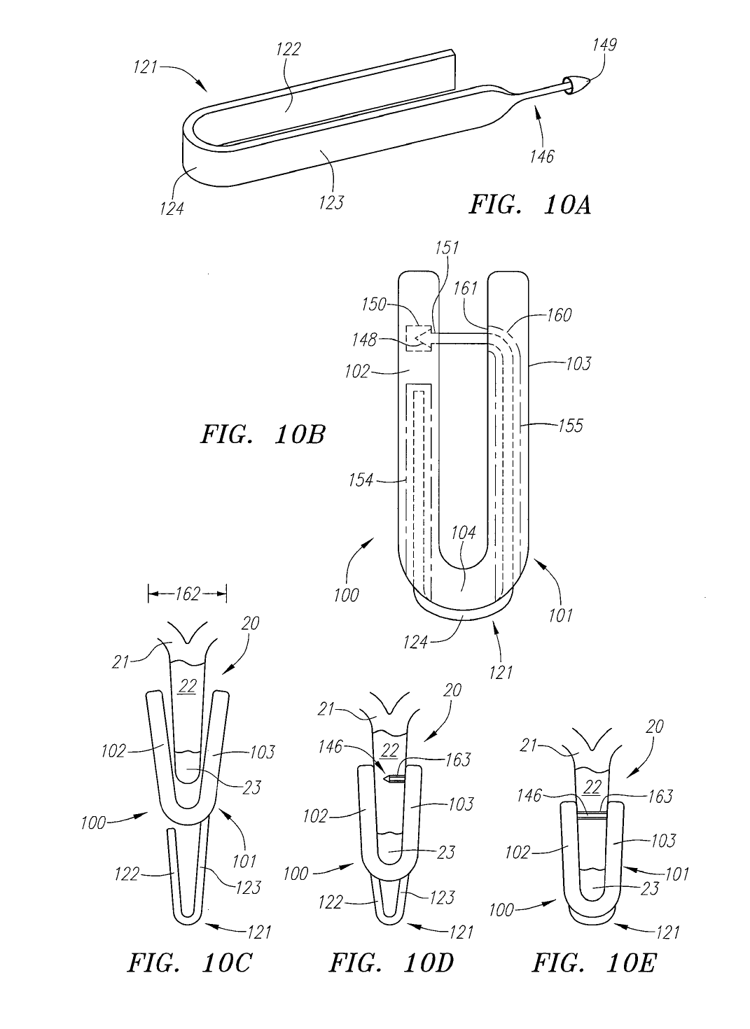

FIGS. 10A-B depict another example embodiment of spacer 100. FIG. 10A is a perspective view of embodiment of closure body 121, while FIG. 10B is a top down view showing spacer 100 in the closed state with closure body 121 positioned within lumens 154 and 155 of main body portion 101. Again, elements that are obscured are shown to reflect this by the use of dashed lines. In this embodiment, closure body right arm portion 123 includes tissue anchor 146 in its distal region. With tissue anchor 146, the entire length of right arm portion 123 is preferably longer than that of left arm portion 122.

Tissue anchor 146 is deflectable from the configuration shown in FIG. 10A to that of FIG. 10B by advancement of closure body 121 through inner lumen 155 of main spacer body right arm portion 103. Lumen 155 includes curved distal portion 160 and open distal end 161, which preferably deflect tissue anchor 146 as it is advanced through that portion of lumen 155. Here, tissue anchor 146 is deflected to an orientation generally perpendicular to the longitudinal axis of right arm portion 123. Open distal end 161 is preferably aligned with recess 150 on left arm portion 102 to guide tissue anchor 146 appropriately. Recess 150 has a relatively narrow opening 151 that acts to retain enlarged head 148 upon advancement therethrough.

In this embodiment, spacer 100 can maintain a relatively low profile and can be implanted without the need to create a wide access opening around the interspinous tissue. FIGS. 10C-E are top down views of an example method of implanting spacer 100 over and through interspinous tissue 20. FIG. 10C depicts body portion 101 during its initial placement prior to the advancement of closure body 121. Here, it can be seen that the initial width 162 of main body portion 101 can be only slightly larger than the width of the interspinous tissue to be engaged. The elimination of a fixed tissue anchor 146 on arm portions 102 or 103 allows the width 162 of body portion 101 to be reduced. Here, closure body 121 has been advanced only partially into main spacer body 101 with left arm portion 102 still remaining outside of inner lumen 154 of left arm portion 102.

FIG. 10D depicts spacer 100 after closure body 121 has been advanced further into main body portion 101. As shown here, tissue anchor 146 has exited right arm portion 103 and has advanced partially through interspinous ligament 22 creating a tissue piercing 163 as tissue anchor 146 travels forward. FIG. 10E depicts spacer 100 once closure body 121 has been advanced the entire way into main body portion 101. Tissue anchor 146 has completely traversed the width of the interspinous ligament 22 and tissue piercing 163 extends entirely therethrough.

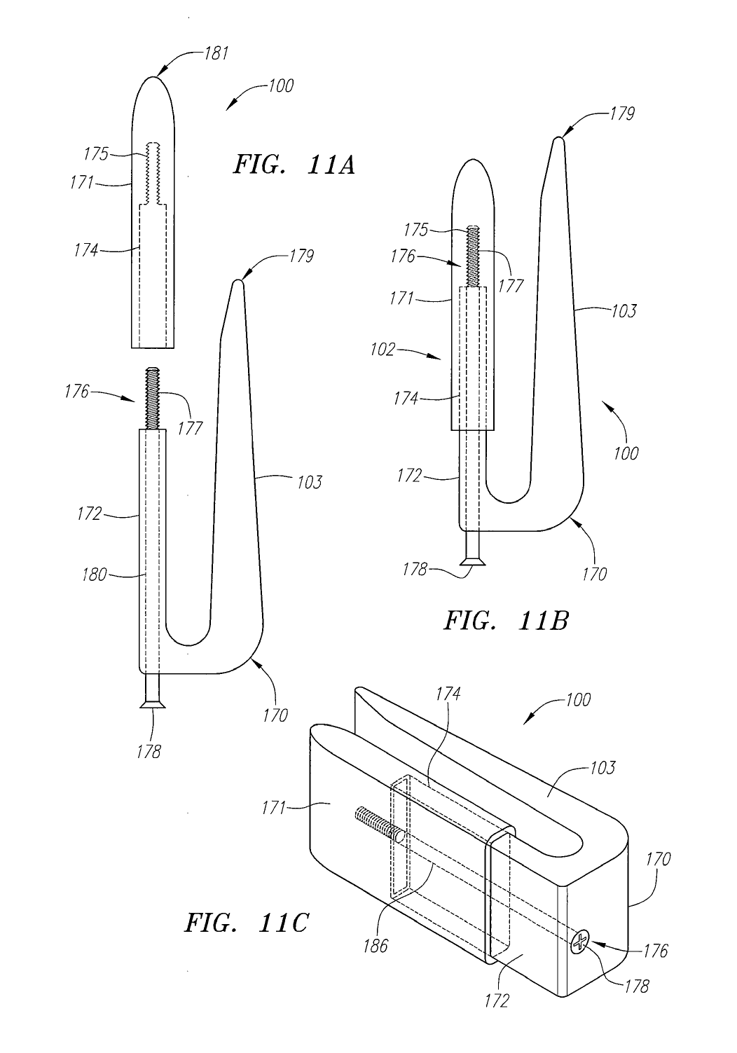

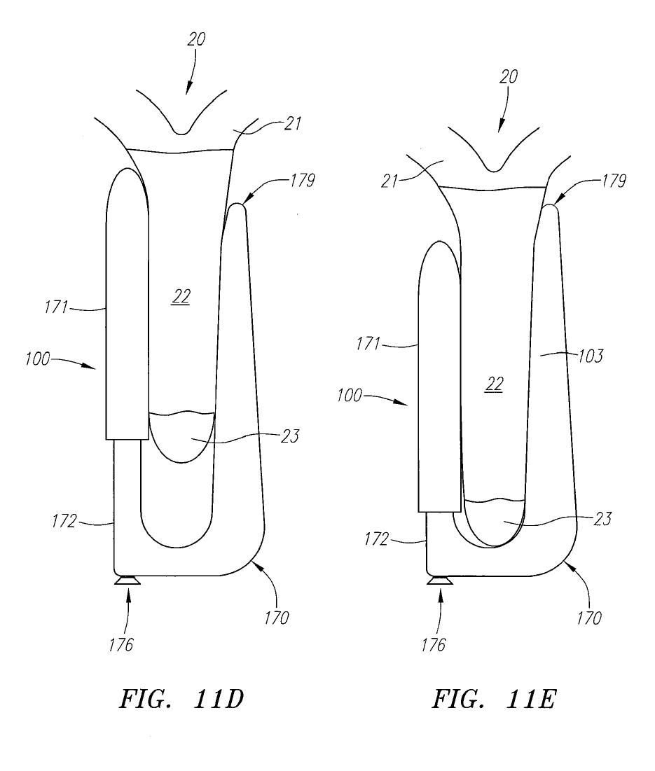

FIGS. 11A-B are top down views depicting yet another example embodiment of spacer 100 in unassembled and assembled states, respectively. Spacer 100 in the assembled state is also shown in the perspective view of FIG. 11C. In this embodiment, spacer 100 includes a first generally U-shaped main body 170 which is connectable to an elongate, plate-like secondary body 171 having a tapered, atraumatic distal end 181. U-shaped body 170 includes right arm portion 103 also having a tapered, atraumatic distal end 179. Body 1 70 also includes an elongate left side portion 1 72 having an attachment mechanism 176 coupled therewith.

In this embodiment, attachment mechanism 176 is an elongate screw rotatably housed within lumen 180. Elongate left side portion 172 is preferably configured to couple with elongate body 171 by sliding into recess 174 of elongate body 171, together forming left arm portion 102. Lumen 174 includes threaded lumen 175 which is preferably configured to receive threaded portion 177 of elongate screw 176. In FIGS. 11B-C, proximal, enlarged head 178 of screw 176 has been rotated to tighten and securely couple bodies 170 and 171 together. It should be noted that any attachment mechanism other than a screw can also be used, including, but not limited to snaps, deflectable tabs, and an interference fit.

FIGS. 11D-E depict the stages of implantation of this embodiment of spacer 100. Preferably, the physician positions body portions 170 and 171, which are connected but not fully tightened together, such that secondary elongate body portion 171 is positioned as desired alongside the interspinous tissue as depicted in FIG. 11D. Once in position, U-shaped body portion 170 can be advanced into the proper position with regards to the interspinous tissue 20 by tightening screw 176 by the appropriate amount. FIG. 11E shows the placement of spacer 100 after the implantation is complete. Similar to the embodiment depicted in FIGS. 10C-E, this embodiment of spacer 100 has a low profile that allows implantation while disturbing only a minimal portion of the body tissue to the left and right side of the interspinous tissue to be engaged.

It should be noted that, as with all embodiments described herein, the features of the embodiment of spacer 100 described with respect to FIGS. 10A-E can be integrated with this embodiment described here with respect to FIGS. 11A-E. Specifically, a slidable tissue anchor can be inserted into a lumen in right arm body portion 103 such that it exits through an open distal end and traverses the interspinous tissue into a recess in left arm portion 102. Alternatively, screw 1 76 can have a deflectable tissue anchor coupled with the screw distal end such that tightening of the screw advances the tissue anchor from within a lumen in elongate portion 171 through the interspinous tissue and, optionally, into a corresponding recess in right arm portion 103.

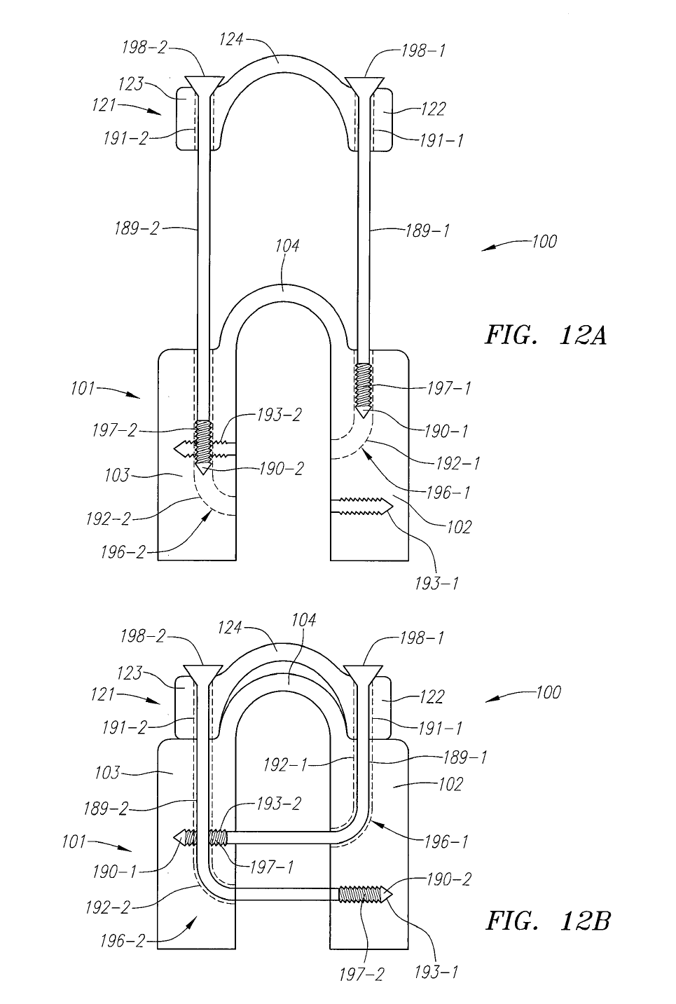

FIGS. 12A-B interior side views of another example embodiment of spacer 100 in partially and fully assembled states, respectively. In this embodiment, closure body 121 is coupled with main spacer body 101 by two deflectable, tissue-piercing attachment mechanisms, or coupling devices 189, which, in this embodiment, are screws. Screws 189 each have a threaded portion 197 and an enlarged head 198 configured to interface with a screwdriver or the like. Located on the distal end of each screw is a substantially sharp distal tip 190 configured to pierce the interspinous tissue. In the partially assembled state of FIG. 12A, screws 189 are housed within inner lumens 191 of closure body 121 and the curved inner lumens 192 of main spacer body 101 (both lumens indicated by dashed lines).

Each curved inner lumen 192-1 and 192-2 has a curved portion 196-1 and 196-2 that are configured to guide deflection of screws 189-1 and 189-2, respectively, as they are advanced towards the assembled state depicted in FIG. 12B. Here, each curved portion 196 is configured to deflect the respective screw 189 by approximately ninety degrees towards the threaded lumen 193 on the opposite side of main body spacer 101. It should be noted that other degrees of deflection can also be used. To allow screws 189 to freely pass each other, the various lumens are preferably staggered in front and back. From the perspective depicted here, lumens 191-2, 192-2 and 193-1 are located in a relatively further forward position than lumens 191-1, 192-1 and 193-2.

To implant this embodiment, spacer 100 is preferably positioned in the desired implant location along the spinal column while in the partially assembled (or similar) state of FIG. 12A. The physician then preferably advances screws 198 within inner lumens 192, through curved portions 196, through the interspinous tissue and into the opposing threaded lumen 193. The amount by which screw 189 is tightened is adjustable and the physician can vary this amount to reach the appropriate degree of closure over and compression of the interspinous tissue. Screw 189 preferably securely engages the opposing arm portions 102 and 103 together, independent of the degree to which it is advanced into lumen 193. Screws 189 can thus act as tissue anchors and adjustable locking devices for maintaining main spacer body 101 in the closed state. It should be noted that any type of closure device including, but not limited to hook-like devices and adjustable locking devices (e.g., zip-tie devices and the like), can be used instead of screws. It should also be noted that closure body 121 can be omitted if desired if other means are used to place left and right arm portions 102 and 103 in the closed state.

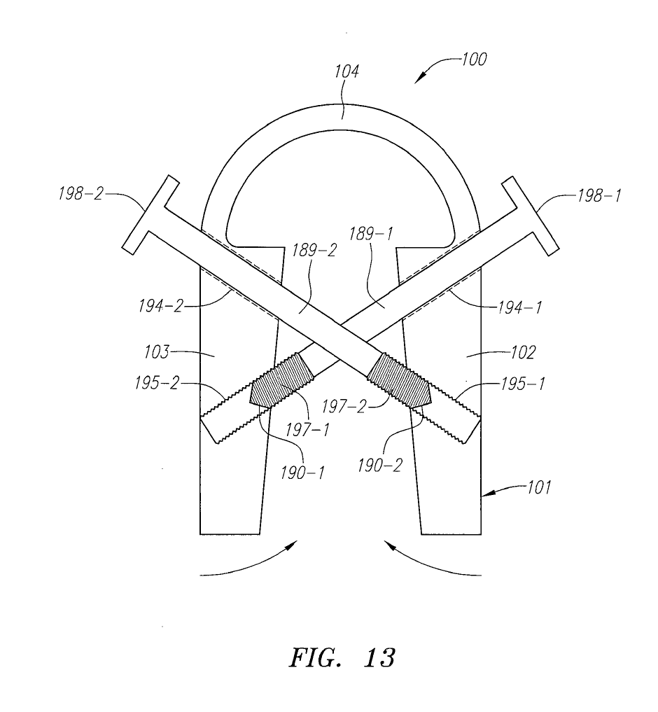

FIG. 13 is an internal side view depicting another example embodiment of spacer 100 having two tissue-piercing coupling devices 189. In this embodiment, no separate closure body is included and coupling devices 189, which are preferably screws, can be used to transition main spacer body 101 into the closed state. Here, both left and right arm portions 102 and 103 each include an angled proximal lumen 194 (indicated by dashed lines) and an angled distal lumen 195, each configured to receive a tissue-piercing screw 189.

After main spacer body 101 is positioned in the desired implant location, screws 189 can be advanced through angled proximal lumen 194, through the interspinous tissue and into the angled distal lumen 195. Angled distal lumen 195 is preferably threaded to receive and securely engage with threaded portion 197 on screw 189. Further advancement of screw 189 into lumen 195 draws left and right arm portions 102 and 103 together towards the closed state. This advancement can cause each lumen 194 to move out of alignment with the opposing lumen 195 and to allow for this, screws 189 are preferably flexible. Alternatively, proximal lumen 194 can be a relatively wide lumen that allows screw 189 to freely move to compensate for this change in alignment.

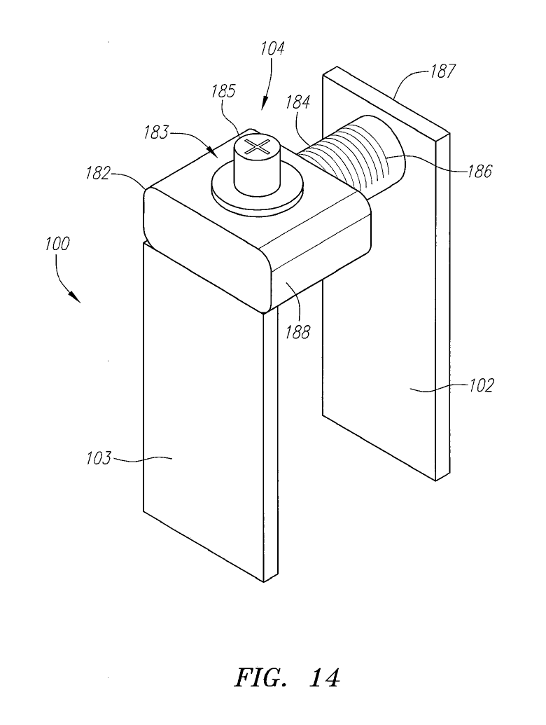

FIG. 14 is a perspective view depicting another example embodiment of spacer 100. Here, spacer 100 includes an adjustably attachable pair of bodies 182 and 187. Body 182 can include right arm portion 103 and a proximally located back-end portion 188, having an adjustable coupling device 183. Body portion 187 can include left arm portion 102 and an elongate, connective strut member 184 configured to interface with coupling device 183 in an adjustable manner.

In this embodiment, bodies 182 and 187 can be ratcheted together by the turning of an actuator 185 on coupling device 183. A plurality of indentations 186 are located on elongate body 184 and are configured to interface with coupling device 183. Indentations 186 provide multiple discrete positions at which bodies 182 and 187 can be coupled together. One of skill in the art will readily recognize the various other mechanisms that can operate as coupling device 183, outside of the rotatable ratcheting system depicted here. For instance, in other embodiments coupling device 183 can be configured as a rack and pinion mechanism, or as any other mechanical coupling system known in the art.

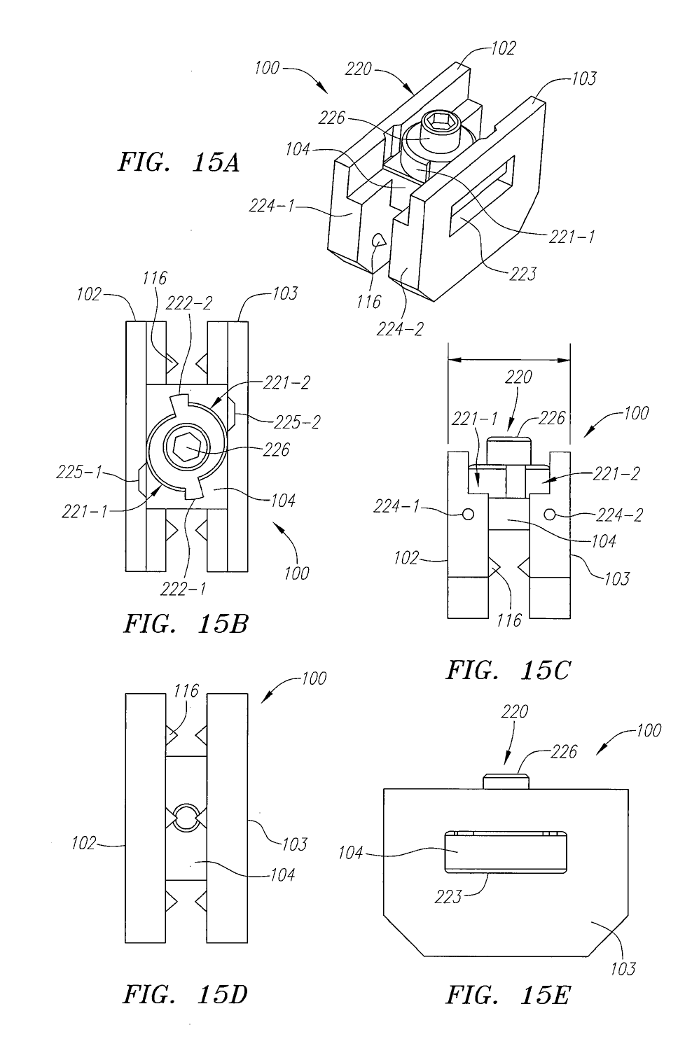

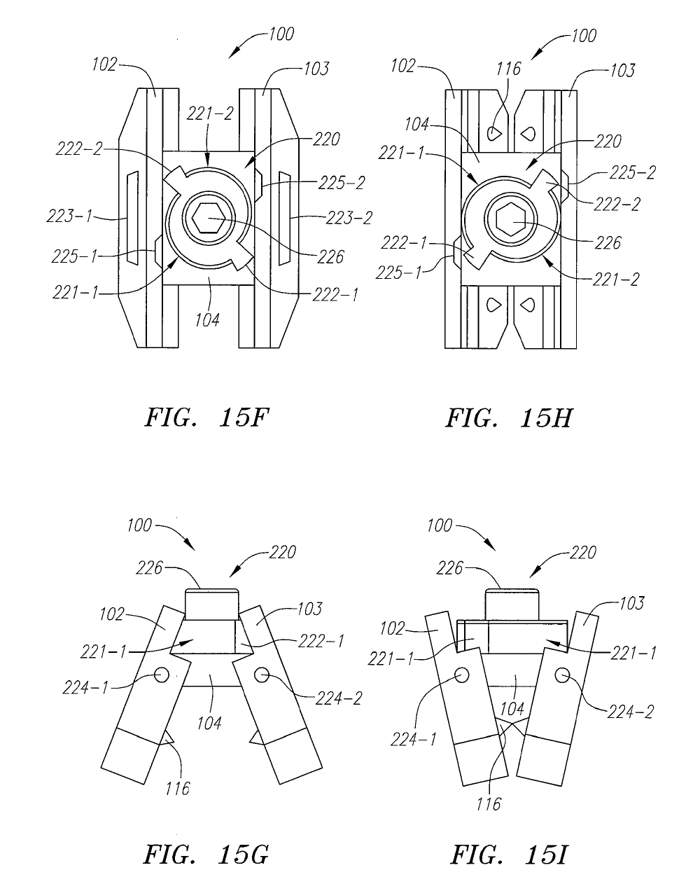

FIGS. 15A-I depict another example embodiment of spacer 100. Here, left arm portion 102 and right arm portion 103 are pivotably coupled with connective portion 104 by way of hinges 224. Coupled with connective portion 104 is a rotatable actuator 220, which is configured to abut left and right arm portions 102 and 103 when rotated and cause portions 102 and 103 to pivot between open and closed states. FIGS. 15A-E depict this embodiment of spacer 100 in an intermediate state approximately halfway between the open state, depicted in FIGS. 15F-G and the closed state, depicted in FIGS. 15H-I. FIG. 15A is a perspective view, FIGS. 15B, 15F and 15H are top down views, FIGS. 15C, 15G and 15I are end-on views, FIG. 15D is a bottom up view and FIG. 15E is a side view of spacer 100.

Actuator 220 includes an interface 226 configured to couple with the delivery device and allow the physician to rotate the actuator 220. Here, interface 220 is a hexagonal recess configured to receive a hexagonal Allen wrench-like member. Each arm portion 102 and 103 also includes an indentation (or recess) 223 configured to allow the delivery device to grasp each arm portion 102 and 103.

The base of actuator 220 includes two curved side surfaces 221-1 and 221-2 located between abutments 222-1 and 222-1, which are oriented approximately 180 degrees apart. Curved side surfaces 221-1 and 221-2 are each preferably placed in contact with the inner face of arm portions 102 and 103, respectively. Each surface 221 preferably has an increasing radius of curvature as measured from the center of rotation of actuator 220. The radius of surface 221-1 is preferably at a maximum adjacent to the abutment 222-1 and likewise for surface 221-2 and abutment 222-2.

Rotation of actuator 220 in a clockwise manner forces the upper (or posterior) sides of arm portions 102 and 103, above hinges 224, in an outward manner causing the lower (or anterior) sides of portions 102 and 103, beneath hinges 224, to pivot towards the closed state. Clockwise rotation is stopped, and the maximum amount of closure is reached, when abutments 222 are fully received into indentations 225 in the left and right arm portions 102 and 103. After positioning spacer 100 in the desired location along the patient's spinal column, the physician can rotate actuator 220 (or cause it to be rotated with the delivery device) to close spacer 100 by the desired amount, up to and including the closed state depicted in FIGS. 15H-I.

As can be seen in FIG. 15F, when in the fully open state, abutments 222-1 and 222-2 are at approximately five o'clock and eleven o'clock positions contacting right and left arm portions 103 and 102, respectively. When fully closed, as depicted in FIG. 15H, abutments 222-1 and 222-2 have been rotated to the approximately eight o'clock and two o'clock positions contacting left and right arm portions 102 and 103, respectively. As one of skill in the art will readily recognize, the amount of rotation necessary to transition spacer 100 between the open and closed states can be varied as desired.

Arm portions 102 and 103 are preferably biased towards the open state such that rotation of actuator 220 in the counterclockwise direction will allow arm portions 102 and 103 to revert to the open state. A spring member (not shown) can be coupled between connective portion 104 and arm portions 102 and 103 to apply the bias, or hinges 224 can be biased towards the open state (e.g., such as by using a nitinol hinge rod and twisting the rod away from its at-rest state as spacer 100 is closed).

The various embodiments of spacer 100 described herein have been shown as having different ratios of height, width and length, both for the overall device and for the various components and portions thereof. These ratios can be varied according to the needs of the individual applications and the embodiments of spacers described herein should not be limited to having any specific ratio between height, width and length and the like, unless expressly set forth in the claims.

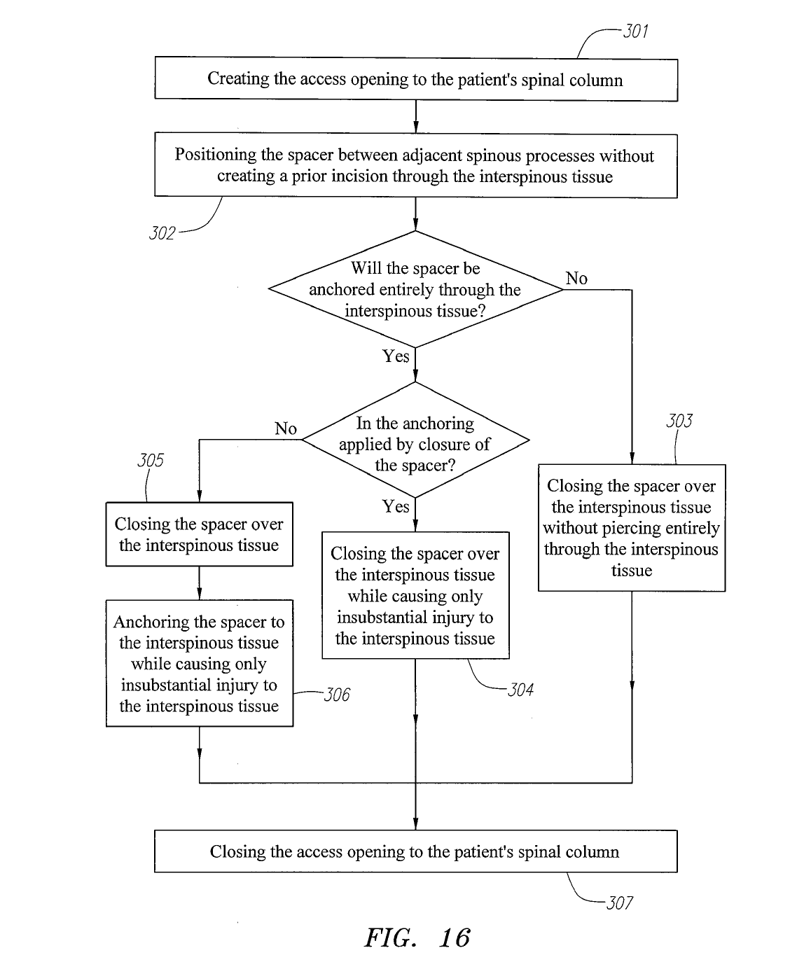

FIG. 16 is a block diagram depicting example methods of implanting any of the embodiments of the spacer described herein. At 301, the physician creates an opening in the patient's back, providing access to the interspinous tissue (e.g., the supraspinous ligament and the interspinous ligament, and also the ligamentum flavum if desired). This may include the application of retaining devices to open the cavity to create sufficient space on both sides of the interspinous tissue to allow the spacer to be positioned. This may also include the removal of excess tissue around the interspinous tissue (e.g., scar tissue, muscle, fatty or bony tissue, etc.) that could prohibit proper device placement.

At 302, the spacer is positioned between the desired adjacent spinous processes. This step can be performed manually by the physician or with the delivery device. It is important to note that the spacer is preferably configured without a trans-interspinal tissue spacing portion such that, prior to placement of the spacer, no incision is required to be made by the physician to create room for implantation of a spacing portion of the device in or through the interspinous tissue. Also, no resection of the interspinous tissue is required to create a cavity in which a spacing portion of the device is to be placed.

If the spacer is to be implanted without the placement of an anchoring means entirely through the interspinous tissue (e.g., a tissue-piercing anchor, screws or sutures, etc.), then, at 303, the spacer is closed over the interspinous tissue (preferably the interspinous ligament and supraspinous ligament) without the presence of, or creation of, any piercing entirely through the interspinous tissue. It should be noted that the presence of one or more tissue engagement features (e.g., 116) may create piercings partially into, but not entirely through, the interspinous tissue. It should also be noted that the spacer can be configured such that it is not required to be closed (see, for example, the embodiment described with respect to FIGS. 11A-E) and merely positioning the spacer over the interspinous tissue is sufficient. In such cases, the closure step can be omitted.

Depending on the implementation, the procedure for closing the spacer will vary. If a closure body is included, then that closure body is applied to the main spacer body, in addition to any closure body retainer. If the spacer includes a closure actuating mechanism, then that mechanism is actuated. If the spacer is self-closable, then removal of any restraint to closure will allow the spacer to self-close. If the spacer is closed with screw-like members, then those screw-like members are tightened. Both the application of the closure body and the actuation of the closure actuator can be performed manually by the physician or with the used of a delivery device.

If the spacer is to be anchored entirely through the interspinous tissue with tissue-piercing anchors fixed to the main spacer body, or fixed to a closure body that is applied to force the main spacer body to close, or through the use of tissue-piercing screws, or the like, then the spacer is closed while causing only “insubstantial injury” to the interspinous tissue, which will be defined below.

If the spacer is to be anchored entirely through the interspinous tissue with sutures or other anchoring means applied after closure of the spacer, then the spacer is preferably first closed at 305, and then anchored to the interspinous tissue at 306 while causing only “insubstantial injury” to the interspinous tissue.

As described, the presence of one or more tissue-piercing anchors (e.g., on the closure body, or the main spacer body, etc.) may pierce entirely through the interspinous tissue, or the physician may apply sutures (or other anchoring means) through the interspinous tissue to lock the spacer in place. However, these anchors/sutures are relatively thin structures that are configured for anchoring the spacer to the spinal column and individually do not act as a trans-interspinal tissue spacing portion, i.e., the load-bearing portion that provides the physical barrier to movement of the adjacent spinous processes towards each other. Because of their small size, these anchors/sutures cause only “insubstantial injury” to the interspinous tissue.

As used herein, “insubstantial injury” refers to the injury caused by one or more relatively small piercings made entirely through the interspinous tissue for the anchoring element. Each small piercing can be made by the physician or by the element itself, or any combination thereof. This insubstantial injury can be contrasted with the substantial injury caused by the following non-exhaustive list of examples: (1) creating an incision into or through the interspinous tissue (e.g., interspinous ligament) to house the load-bearing or spacing portion of the device; or (2) resecting or removing interspinous tissue (e.g., interspinous ligament) to create a cavity for housing the load-bearing or spacing portion of the device.

After closure of the spacer, the access opening to the spinal column can be closed at 307, at which point the implantation of the device is preferably complete.

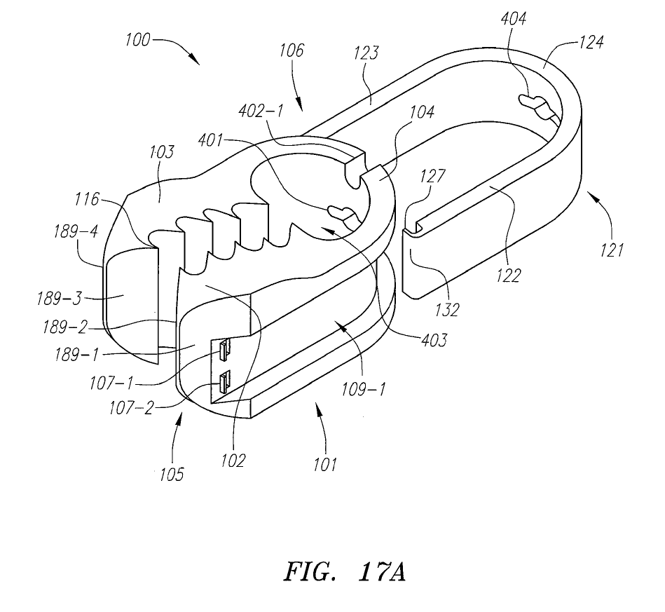

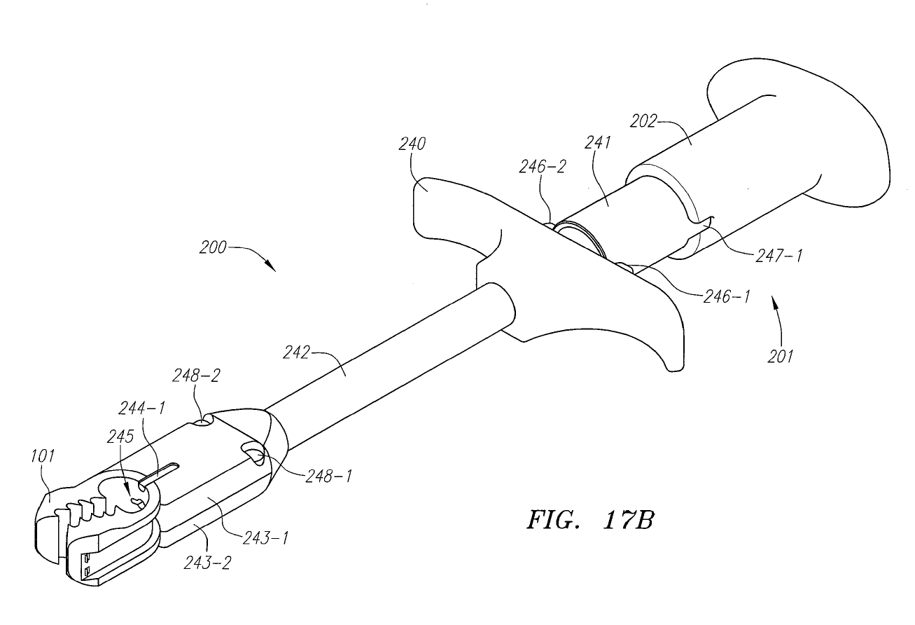

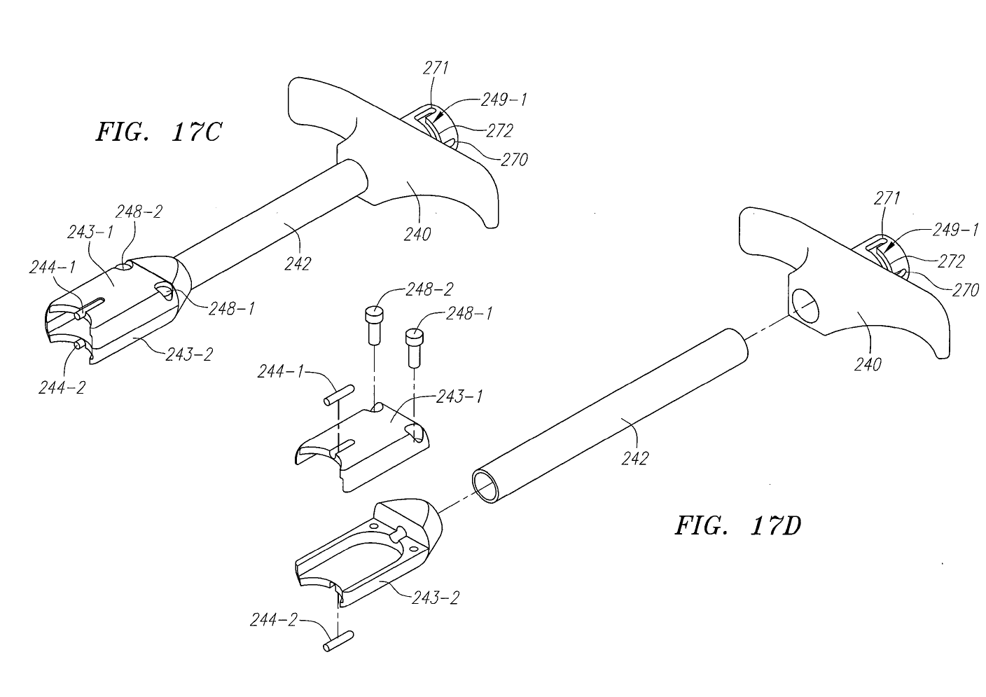

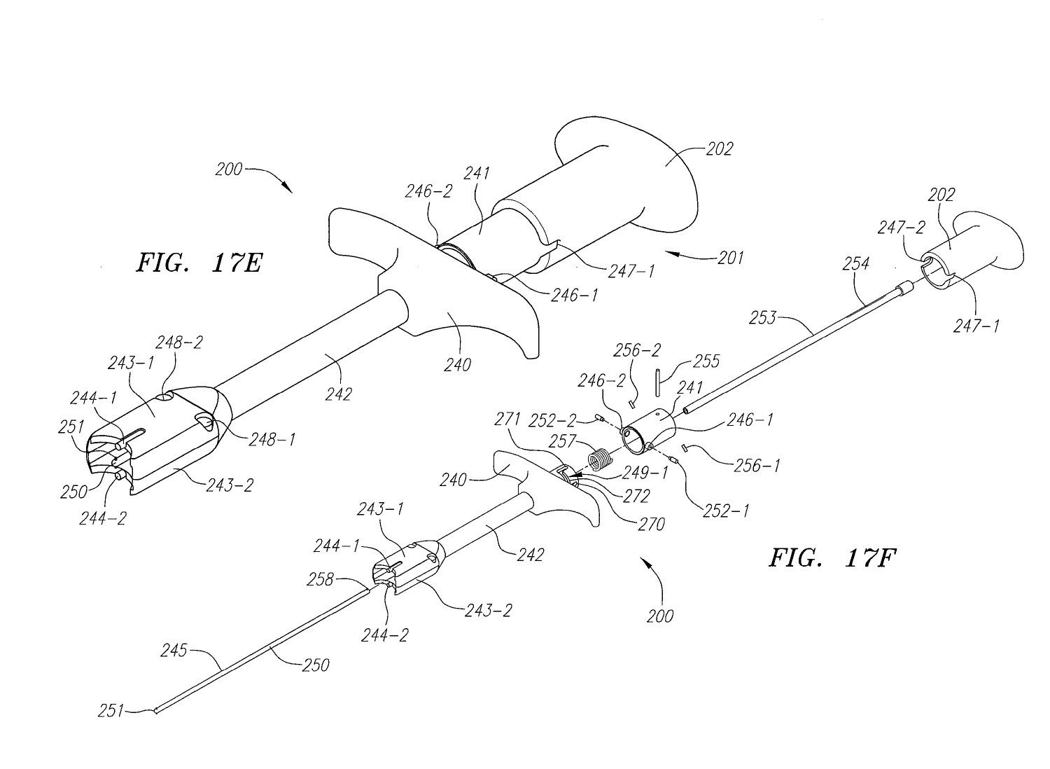

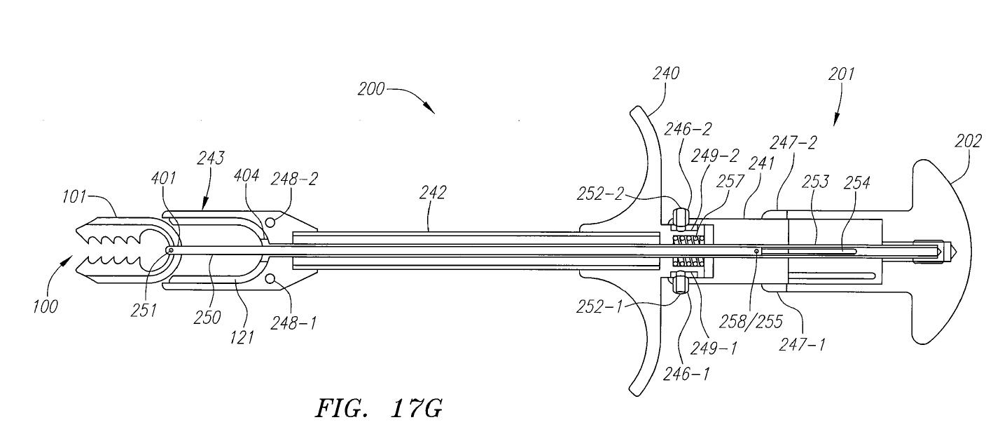

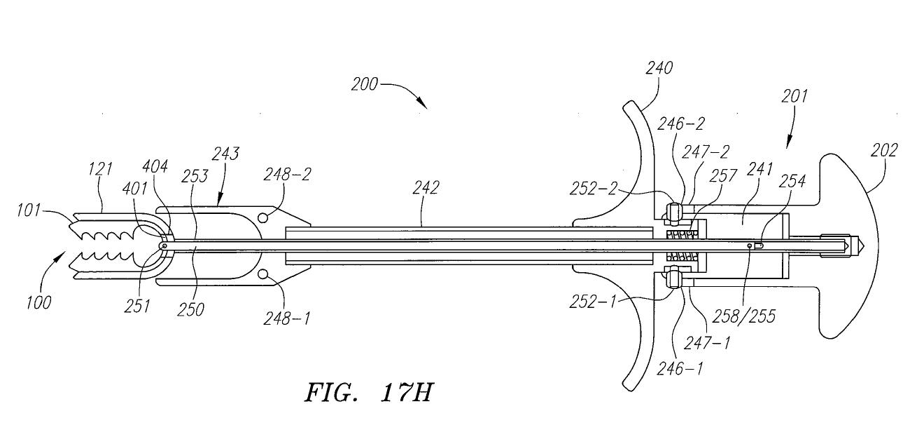

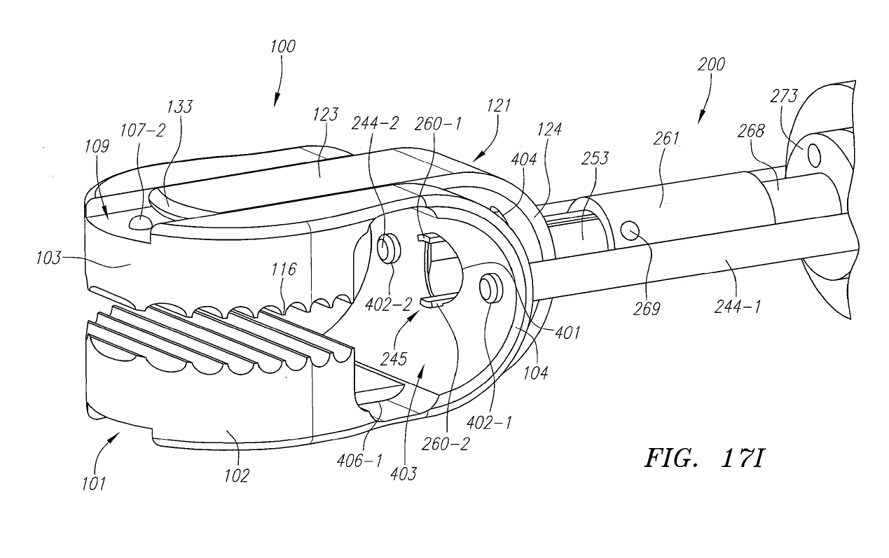

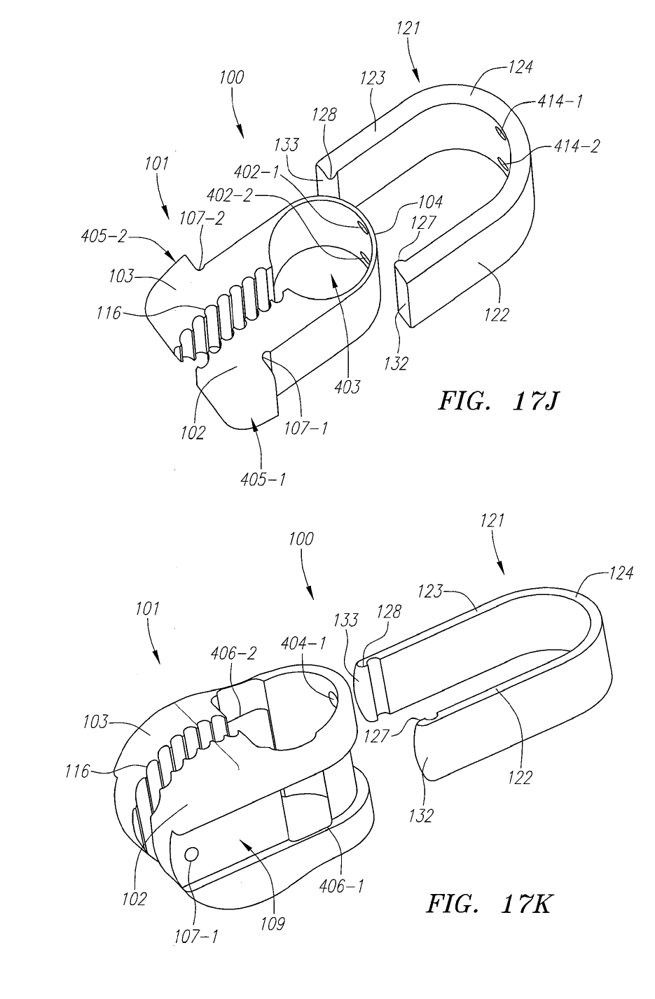

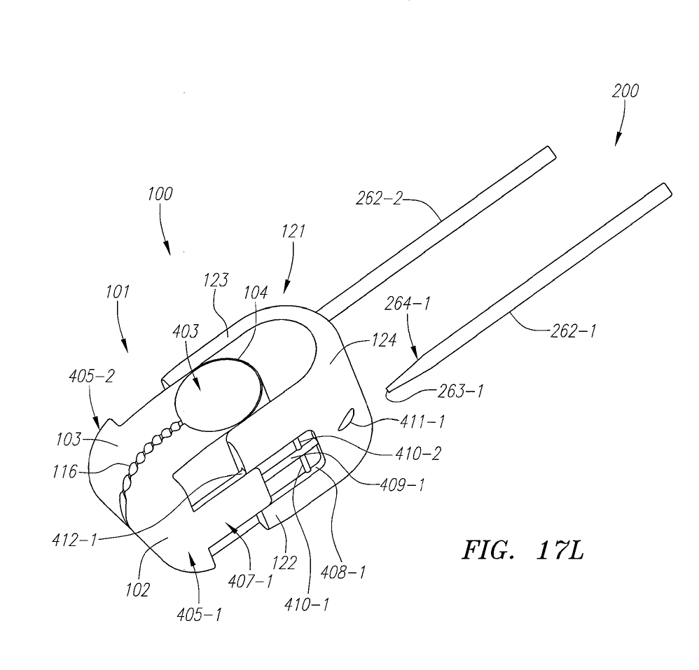

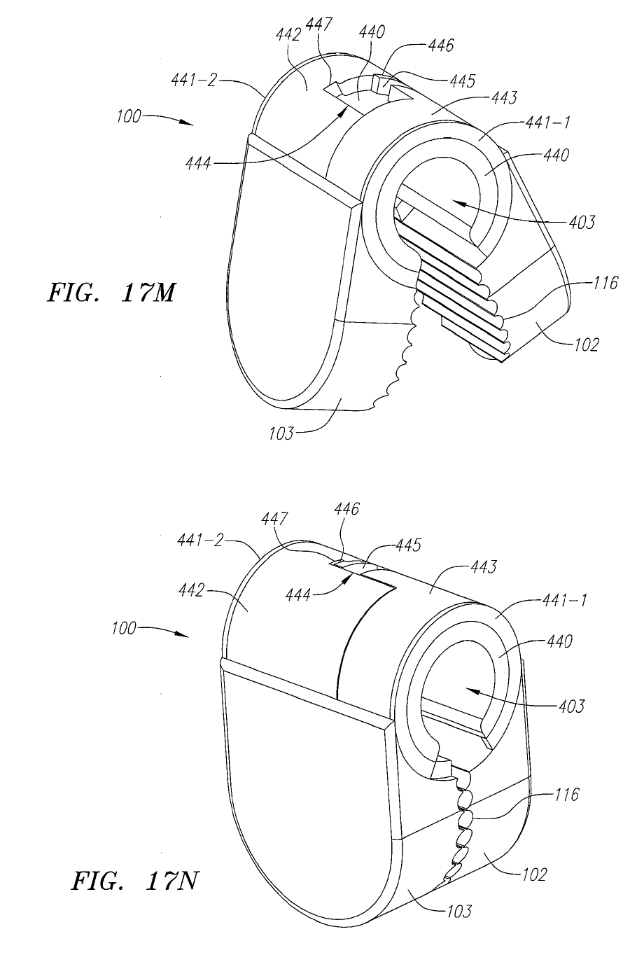

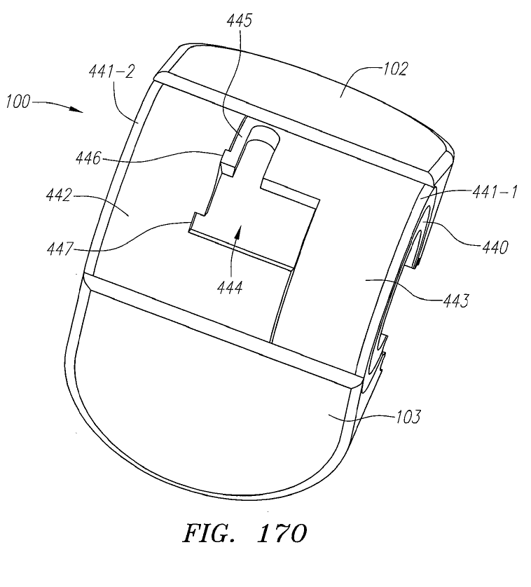

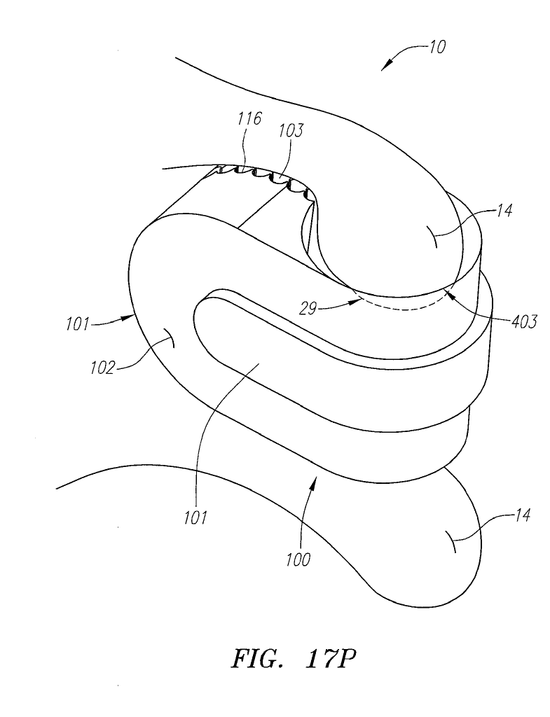

FIG. 17A depicts another example embodiment of spacer 100 having a U-shaped main spacer body 101 configured to slidably receive a closure body 121 having a generally complementary U-shape. Here, left arm portion 102 of main spacer body 101 includes interface features 107-1 and 107-2 which form abutments over which abutment 127 on closure body 121 can slide to retain closure body 121 in place. Main spacer body 101 includes an open region 403 towards its proximal end 106 to accommodate interspinous tissue such as the supraspinous ligament. Here, open region 403 is a generally cylindrical lumen separating arm portions 102 and 103. This lumen has a width that is relatively greater than a width (or all widths) between arm portions 102 and 103 when in the closed state. Viewed from a different perspective, arm portions 102 and 103 have relatively thicker sidewalls in the region corresponding to engagement with the interspinous ligament (e.g., adjacent tissue engagement features 116 described below). Proximal to this region, arm portions 102 and 103 have relatively thinner sidewalls with the same thickness as connective portion 104. Open region 403 can allow spacer 100 to rest over the supraspinous ligament while minimizing any trauma thereto. If region 403 is configured with a relatively large width, some limited motion of the supraspinous ligament therein is achievable. Preferably, region 403 is large enough to avoid the application of significant, if any, compressive force on supraspinous ligament.

Also present are a plurality of tissue engagement features 116, each of which has a tooth-shaped configuration inclined proximally to resist being pulled off. (It should be noted that other configurations of engagement features 116, such as the overlapping teeth-like elements described with respect to FIGS. 18A-B, can also be used.) In a preferred embodiment, the tissue engaging portion of spacer 100, which are tissue engagement features 116 in this embodiment, are configured to engage with only the interspinous ligament and not the supraspinous ligament, which is contained within open region 403, or the ligamentum flavum, which is preferably anterior (or distal) to tissue engagement features 116. Such a configuration provides targeted engagement of spacer 100 with only the interspinous ligament and minimizes trauma or irritation to the supraspinous ligament and ligamentum flavum. To minimize trauma/irritation to the supraspinous ligament, the portion of main spacer body 101 adjacent open region 403 can be fabricated from or coated with suitable atraumatic materials, e.g., materials that inhibit inflammatory response, lubricious materials, and the like.

A release mechanism receiving slot 401 is present in main spacer body 101 and a corresponding slot 404 is present in closure body 121. Also shown here is an upper aperture 402-1 (a corresponding lower aperture is obscured). The functions of slots 401 and 404 and aperture 402 will be described in more detail. The distal ends of arm portions 102 and 103 can be tapered or beveled to ease insertion of the device into the desired location, and to also dissect through tissue disposed laterally adjacent to the interspinous and supraspinous ligaments. Here, arm portion 102 includes two opposing distal bevels 189-1 and 189-2 and arm portion 103 includes two opposing distal bevels 189-3 and 189-4. Although two bevels 189 are present on each arm portion, any number of one or more can be used.

FIG. 17B is a perspective view of a delivery system 200 configured to deliver the embodiment of spacer 100 shown and described with respect to FIG. 17A. Delivery system 200 includes proximal handle portion 201 having an actuator 202, a forward grip 240 and a proximal release interface 241. Forward grip 240 is coupled with a shaft 242 that is in turn coupled with a shaft base housing 243, which, in this embodiment, includes upper shaft base housing 243-1 and lower shaft base housing 243-2. Shaft base housings 243-1 and 243-2 are coupled together by coupling devices 248-1 and 248-2, which, in this embodiment, are configured as screws. Main spacer body 101 is coupled with the distal end of shaft base housing 243 and is held in place by a release mechanism 245, which will be described in more detail. A seat 244 for main spacer body 101 is formed by rods 244-1 and 244-2 (rod 244-2 is obscured in this view), which interface with apertures 402-1 and 402-2, respectively, on main spacer body 101.

Generally, at the appropriate location along the patient's spinal column, delivery system 200 is used to deliver main spacer body 101 in a position that will distract the adjacent spinous processes. Once properly positioned, actuator 202 is advanced distally to force closure body 121 (not shown) over main spacer body 101 and into the locked position. Advancement of actuator 202 continues until slots 247-1 and 247-2 (247-2 is obscured in this view) receive outer abutments 246-1 and 246-2, respectively, located on proximal release interface 241. Here, abutments 246-1 and 246-2 are configured as rods. Proximal release interface 241 is coupled with release mechanism 245 and is rotatable to rotate release mechanism 245 into a position that allows the release of spacer 100. This is accomplished by rotation of actuator 202, which in turn rotates release mechanism 241 through the interface of rods 246 and slots 247.

FIG. 17C is a perspective view of a portion of delivery system 200 including forward grip 240, shaft 242 and shaft base housings 243-1 and 243-2. FIG. 17D is an exploded view of the embodiment depicted in FIG. 17C showing the components in more detail. Again, upper shaft base housing 243-1 is coupled with lower shaft base housing 243-2 by screws 248-1 and 248-2. Rods 244-1 and 244-2 are coupled with the distal end of shaft base housings 243-1 and 243-2 and act as a seat for main spacer body 101 (not shown). Shaft base housing 243 is coupled with shaft 242, which in turn is coupled with forward grip 240. At the proximal end of forward grip 240 is a cylindrical section having slots 249-1 and 249-2 (slot 249-2 is obscured on the opposing side) that act as a guide for the release interface 241 (not shown). Slots 249 each include two longitudinal track portions 270 and 271 that correspond to the locked and unlocked positions of release mechanism 245. A lateral track portion 272 separates portions 270 and 271

FIG. 17E is another prospective view of delivery system 200 shown without spacer 100 attached at its distal end. FIG. 17F is an exploded view showing the components of delivery system 200 in more detail. Referring to FIG. 17F, portions shown and described with respect to FIGS. 17C-E including forward grip 240, shaft 242 and shaft base housing 243 are shown along with the remaining components of system 200. A push tube 253 having a longitudinal slot 254 is coupled with actuator 202 such that distal and proximal motion of actuator 202 causes push tube 253 to move in a likewise manner. Push tube 253 preferably slides within proximal release interface 241, which in this embodiment is cylindrical with an inner lumen. Inner abutments 252-1 and 252-2 are coupled with release interface 241 and extend into the inner lumen of interface 241. Here, inner abutments 252 are configured as rods and slide within guide slots 249-1 and 249-2, respectively (guide slot 249-2 is obscured in this view).

A bias element 257 is located between forward grip 240 and actuator 202 and biases actuator 202 to the extended proximal position shown in FIG. 17E. In this embodiment, bias element 257 is configured as a spring. A crossbar 255 is placed within slot 254 of push bar 253 and both guides advancement of push bar 253 and couples actuator 202 to the assembly. Release mechanism 245 includes a shaft 250 and a distal portion having opposing abutments. Here, the opposing abutments are formed by a T-bar 251 extending through shaft 250. Shaft 250 includes an aperture 258 at its proximal end through which crossbar 255 is inserted to lock release mechanism 245 in position with respect to release interface 241. In this manner, rotation of release interface 241 causes corresponding rotation of release mechanism 245. Referring back to FIG. 17A it can be seen that slots 401 and 404 have a configuration shaped to receive shaft 250 with T-bar 251 at only certain rotational orientations. T-bar 251 is preferably oriented as shown in FIGS. 17E-F to lock spacer 100 against shaft base housing 243. Rotation of T-bar 251 by approximately 90 degrees aligns T-bar 251 with slots 401 and 404 and allows the release of spacer 100.

FIGS. 17G-H are cross-sectional views of delivery system 200 and spacer 100 during delivery. FIG. 17G depicts system 200 and spacer 100 in the configuration suited for initial placement of main spacer body 101 between adjacent spinous processes, while FIG. 17H depicts main spacer body 101 after closure body 121 has been advanced to lock spacer 100 onto the interspinous tissue. It should be noted that main spacer body 101 can initially assume an open configuration in FIG. 17G, with arm portion 102 and 103 spaced relatively more so than depicted, to allow spacer 100 to be more easily positioned over the interspinous tissue. Advancement of closure body 121 over main spacer body 101 causes main spacer body 101 to transition to the closed state, e.g., to clamp main spacer body 101 onto the interspinous tissue.

In FIG. 17G, closure body 121 is shown located within shaft base housing 243. The distal end of pusher tube 253 is disposed adjacent to closure body 121 and actuator 202 is in the proximal position prior to advancement. Bias element 257 is shown in an extended state exerting a proximal force against release interface 241 to maintain T-bar 251 pressed securely against the backside of main spacer body 101.

FIG. 17H depicts actuator 202 after being advanced distally such that slots 247 receive rods 246. Advancement of actuator 202 into this configuration has advanced closure body 121 from within shaft base housing 243 and over main spacer body 101. Further advancement of actuator 202 causes rods 246 to be received further within slots 247 and causes the compression of bias element 257. Inner rods 252 are preferably located in longitudinally track portion 270 of slots 249. Longitudinally track portion 270 prevents rotation of release interface 241 prior to release of spacer 100.

Continued advancement of actuator 202 causes the compression of bias element 257 and slides release interface 241 distally to the base of the longitudinal track portion 270 adjacent lateral track portion 272 of slot 249 to allow rotation of release interface 241. This advancement also moves release shaft 250 distally causing T-bar 251 to be transitioned into open region 403 of main spacer body 100. At this point, actuator 202 can be rotated such that rods 252 are moved through lateral track portion 272 to longitudinal track portion 271. This rotation of actuator 202 causes corresponding rotation of T-bar 251 such that it is aligned with slots 401 and 404 of spacer 100. Bias element 257 will force actuator 202 in a proximal direction to retract T-bar 251 through slots 401 and 404 in main spacer body 10 and closure body 121, respectively. This action releases spacer 100 from delivery system 200.

FIG. 17I is a perspective view depicting the distal portion of a delivery system 200 having a spacer 100 coupled thereto and retained by release mechanism 245. In this embodiment, release mechanism 245 is a pair of deflectable struts 260-1 and 260-2. Struts 260 each have an abutment on their distal ends that is configured to engage with the edge of spacer body 101 adjacent aperture 401. The proximal end (not shown) of struts 260 are integrally coupled with outer tubular member 268, which in turn is coupled with hub 273 of system 200. Deflection of struts 260 towards each other disengages the abutments from spacer body 101 and allows release therefrom. Struts 260-1 and 260-2 are biased towards the outwardly deflected configuration depicted here. In this embodiment, the outer surface of struts 260 are tapered such that the strut thickness increases from the proximal end to the distal end. Release occurs by advancement of collar 261 over struts 260 such that the struts are deflected towards each other to disengage the distal abutments from spacer body 101. Alternatively, the inner surface of collar 261 can be tapered to achieve a similar result. A coupling pin 269 couples collar 261 with an inner tubular member (not shown) that preferably resides between pusher member 253 and outer tubular member 268. The inner tubular member is slidable with respect to outer tubular member 268 and pusher member 253 to allow collar 261 to be independently advanced and retracted as desired.

Also of note, closure body 121 includes aperture 404 which is configured to allow struts 260 to deflect back and forth and pass therethrough. Thus, to deploy spacer 100, closure body 121 is first advanced into position by pusher tube 253. After placement of closure body 121 engagement device 245 can be released by the advancement of collar 261.

FIG. 17J depicts another example embodiment of spacer 100. Here, each arm portion 102 and 103 has a relatively enlarged base portions 405-1 and 405-2, respectively. Adjacent to base portions 405 are indentations 107-1 and 107-2 that form interface features for interfacing with corresponding abutments 127 and 128 on closure body 121. Apertures 102-1 and 102-2 are shown in connective portion 104 of main spacer body 101 and corresponding apertures 414-1 and 414-2 are shown in connective portion 124 of closure body 121. Not shown here are slots 401 and 404 for engaging with the release mechanism. It should be noted that these slots, as well as apertures 402 and 414, are optional and can be incorporated depending on the configuration of the delivery system.

FIG. 17K is a perspective view showing another example embodiment of spacer 100. Arm portions 102 and 103 can include window-like apertures 406-1 and 406-2, respectively. Closure body 121 is configured to slide within guide track 109 until abutments 127 and 128 are advanced over interface features 107-1 and 107-2 (feature 107-2 is obscured in this view).

FIG. 17L is a perspective view depicting another example embodiment of spacer 100 as well as prong-like members 262, which can be configured as part of delivery system 200 or a separate removal tool. Here, spacer 100 is configured to facilitate removal of closure body 121 from the secured position. The medical professional has the capability to readily unlock closure body 121 from main spacer body 101 should the device need to be removed, repositioned or otherwise redeployed.

Here, left arm portion 102 includes a flap-like outer strut 407-1 configured to slide into a recess 408-1 in closure body arm portion 122. Recess 408-1 is configured to guide the relative movements of spacer body 101 and closure body 121. The inner surface of strut 407-1 can include one or more indentations 412-1 for receiving corresponding abutments 410-1 and 410-2 on the outer surface of left arm portion 122. These corresponding features, when engaged, provide a means for securement of the two bodies 101 and 121 relative to each other. An aperture 411-1 is present towards the proximal connective portion 124 of closure body 121. This aperture provides entrance to a lumen that extends towards recessed portion 408-1. This lumen is formed along the length of arm portion 122 by an elongate, semi-circular recess 409-1 and along the inner surface of strut 407-1 by a corresponding recess elongate, semi-circular recess (not shown). Similar features are preferably also present on the right side of spacer 100.

Apertures 411 are configured to each receive a prong-like member 262, the proximal portions of which can be coupled with delivery system 200, or a removal tool, or prong-like members 262 can be used as individually as separate devices. Each prong-like member 262-1 preferably has a similar configuration, for instance, prong-like member 262-1 preferably has a tapered end portion 264-1 with a pointed distal end 263-1. Recess 409-1 and the corresponding recess on the inner surface of strut 407-1 are preferably sized to receive the tapered portion 264-1 of prong-like member 262-1. However, the width of recess 409-1 and the corresponding recess on strut 407-1 is preferably not sufficient to accommodate the width of member 262 at its widest point. Thus, continued advancement of member 262-1 into aperture 411-1 causes strut 407-1 to deflect outwards and disengage indentation 412-1 from abutments 410-1 and 410-2. In this manner, members 262 can be used to disengage each strut 407 on main spacer body 101 from closure body 121. Closure body 121 can then be proximally retracted with respect to main spacer body 101 to remove it therefrom. Prongs 262 can be configured with a latch mechanism to engage closure body 121 after being unlocked from main spacer body 101 to facilitate retraction of closure body 121.

FIGS. 17M-O are perspective views depicting another example embodiment of spacer 100. Here, spacer 100 is configured to function with a self-locking feature to lock the device in the appropriate closed state. Spacer 100 includes left arm portion 102, right arm portion 103, and a proximal tubular member 440 having flanged ends 441-1 and 441-2. Arm portions 102 and 103 are configured to swivel from the open configuration (depicted in FIG. 17M) to the closed configuration (depicted in FIG. 17N) about tubular body 440. Arm portion 102 includes a proximally positioned curved member 442. Curved member 442 is sheet-like and fits closely over tubular member 440 to maintain a low profile. Arm portion 103 also contains a generally sheet-like curved member 443. Members 442 and 443 are preferably shaped in a complementary fashion, which is a stepped, or L-shaped, configuration in this embodiment. L-shaped members 442 and 443 are depicted in more detail in the perspective view FIG. 17O (showing the proximal side of spacer 100 while in the open state of FIG. 17M).

A gap 444 exists between L-shaped members 442 and 443 to allow transition from the open to the closed state. L-shaped member 443 preferably includes a deflectable arm member 445 having a catch 446. In FIG. 17O, deflectable arm member 445 is deflected and biased to return to its at-rest state. As arm portions 102 and 103 are advanced towards each other to close spacer 100, catch 446 moves over detent 447 in curved member 442, at which point deflectable arm member 445 can deflect and advance catch 446 into detent 447 to lock spacer 100. Here, catch 446 has a stepped configuration, but can have other configurations (e.g., rounded, sawtooth and the like) that facilitate release should the medical professional desires to again open spacer 100. Thus, spacer 100 can be repeatedly transitioned between open and closed configurations as many times as is desired by the medical professional.

Although only one detent 447 is shown in this embodiment, it should be noted that multiple detents 447 can be positioned to allow spacer 100 to be adjusted amongst incremented lock positions (e.g., in a ratchet-like fashion).

As noted, open region 403 of spacer 100 accommodates the supraspinous ligament. Region 403 can also be configured to accommodate an inferiorly extending portion 29 of the superiorly located spinous process, as depicted in the perspective view of FIG. 17P. Such superiorly extending portions 29 are typically most pronounced in the thoracic region of the spine, but can also be present in the cervical and lumbar regions. Here, the superiorly extending portion 29 located within open region 403 is indicated as being obscured by main spacer body 101 by the dashed lines. Also, the interspinous tissue other than the spinous processes 14 is not shown. Placement of spacer 100 over spinous process 14 can allow the placement of a relatively larger spacer 100 and reduce the risk that spacer 100 could rotate superiorly or inferiorly within the interspinous space.

FIGS. 18A-B depict additional example embodiments of spacer 100. Referring first to FIG. 18A, spacer 100 has a multi-piece construction and includes a mechanical actuator 425 for controlling the opening and closing of the device. Here, arm portions 102 and 103 have divided proximal ends with an open region 426 therebetween. A central, curved strut 417 is located within open region 426 and pivotably mounted on arm portions 102 and 103 with hinges 416-1 and 416-2, respectively. It should be noted that arm portions 102 and 103 can move apart in any desired manner, such as sliding, ratcheting, translating, rotating, expanding and the like. A second central, curved strut 418 is located proximal to strut 417 within open region 426 and has opposing guide rods 419-1 and 419-2 extending therefrom into corresponding slots 420-1 and 420-2 on arm portions 102 and 103, respectively. Rods 419 and slots 420 are configured to guide motion of arm portions 102 and 103 between the closed and opened states.

Actuator 425 is located on proximal end 106 of spacer 100. Here, actuator 425 is configured as a screw (with any desired interface) rotatably received within a threaded slot in strut 418. Rotation of actuator 425 causes strut 418 to move relative to strut 417. Proximal movement of strut 418 causes spacer 100 to transition towards the closed state due to the movement of guide rods 420 within slots 420. Conversely, distal movement of strut 418 relative to strut 417 causes spacer 100 to transition to the open state. It should be noted that any other type of actuation mechanism other than screws can be used.

Arm portions 102 and 103 also include complementary tissue engagement features that are configured to trap the interspinous tissue. For instance, arm portion 102 includes teeth-like outcroppings 421-1 and 421-2 while arm portion 103 includes tooth-like outcropping 421-3, each of which have similar rounded box-like or block-like configurations. Teeth 421 are configured for receipt within corresponding recesses 423. A larger tooth-like outcropping 428 is located on the distal end of arm portion 103, which is configured to be received within recess 429 on arm portion 102. Closure of arm portions 102 and 103 preferably forces the interspinous tissue to comply with the tissue engagement features. This can trap the interspinous tissue between arm portions 102 and 103 and securely lock spacer 100 in place.

This configuration is preferably used in an application where the patient's interspinous tissue is relatively thin and flexible such that it can be molded or displaced yet would allow arm portions 102 and 103 to adequately close. The degree of overlap between tissue engagement features allows for a greater engagement with the patient's interspinous tissue and also acts to counter any tendency spacer 100 would have to rotate inferiorly or superiorly with respect to the patient's spinal column. In addition, overlapping tissue engagement features make it difficult for spacer 100 to be inadvertently forced open, such as by one or more of the adjacent spinous processes during extension of the spine.

FIG. 18B depicts another example embodiment similar to that shown and described with respect to FIG. 18A. Here, arm portions 102 and 103 include extended tooth-like segments 430-1 and 430-2, respectively. Segments 430-1 and 430-2 are configured for receipt within opposing recesses 431-1 and 431-2. Like the previous embodiment, this configuration can be used in an application when the patient's interspinous tissue is relatively thin and flexible and will distend to the degree necessary to accommodate segments 430. It should be noted that the complementary teeth-like elements described with respect to FIGS. 18A-B can likewise be used with any of the embodiments of spacer 100 described herein.

FIGS. 18C-D are perspective views depicting an additional example embodiment of a spacer 100 having a multi-piece construction. Like the embodiments described with respect to FIGS. 18A-B, this embodiment of spacer 100 includes a mechanical actuator 425 for controlling the opening and closing of arm portions 102 and 103. It should be noted that other forms of actuation, such as electrical, magnetic, thermal (e.g., shape memory) and the like, can also be used.

Also similar to the previous embodiments, spacer 100 includes block-like outcroppings 421 (e.g., teeth) that are configured to engage tissue and are received within corresponding recesses 423. These engagement features are located in the relatively thick distal section adjacent distal end 105. This relatively thick distal section is preferably used for targeted engagement of only the interspinous ligament. A distal portion of each arm portion 102 and 103 has a raised upper surface 490 such that it flares upward when proceeding away from the center portion of spacer 100 (or tapers downward when proceeding in the opposite direction). This raised upper surface 490 is configured to interface with the shape of the spinous process, and will be discussed in more detail with regard to FIGS. 18L-K. Each arm portion 102 and 103 has a beveled distal side surface 491, which can aid in insertion of spacer 100 into the interspinous space. An open region 403 is located proximal to these engagement features to accommodate interspinous tissue such as the supraspinous ligament.

Spacer 100 can be transitioned between the open and closed configurations by manipulation of actuator 425. Actuator 425, in this embodiment, is a rotatable element 453 with an enlarged proximal end. Arm portions 102 and 103 are coupled together at the proximal side of spacer 100 by hinge members 452-1 (shown) and 452-2 (not shown). Hinge 452 allows arm portions 102 and 103 to pivot with respect to each other.

A proximally-located member 455 is coupled with each of arm portions 102 and 103 such that member 455 is moveable in relation to arm portions 102 and 103. Here, the proximal member 455 is U-shaped and configured as a cover, casing or shroud, with struts that overlap at least part of the proximal end of arm portions 102 and 103. Proximal member 455 can have any desired configuration and is not limited to a U-shape. A recess or gap 456 is located adjacent each arm portion 102 and 103 and allows movement of arms 102 and 103 between the open and closed configurations. Guide rods 450 are preferably fixed on both sides of U-shaped member 455 and are located through an elongate slot 451 in each arm portion 102 and 103. Movement of U-shaped member 455 distally and proximally with respect to arm portions 102 and 103 within elongate slots 451-1 and 451-2, respectively, forces arm portions 102 and 103 to move with respect to each other.

Advancement of U-shaped member 455 towards arm portions 102 and 103 causes spacer 100 to close as depicted in FIG. 18D. This is due, in part, to the orientation of elongate slots 451 in a manner that extends generally longitudinally with each arm portion 102 and 103. Movement of U-shaped member 455 with respect to arm portions 102 and 103 can be achieved by rotatable member 453, which in this embodiment is configured as a threaded bolt with a polygonal bolt head. One of skill in the art will readily recognize that numerous other elements, not limited to rotatable elements, can also be used as actuator 425. Rotatable member 453 includes an internal lumen 454 for interfacing with a delivery system 200 as will be described later.

FIGS. 18E-F are top-down views of this embodiment of spacer 100 in the open and closed configurations, respectively. Here, the use of rotatable member 453 to cause spacer 100 to transition between the open and closed configurations is shown in more detail. A generally cylindrical interface member 456 is preferably located between hinge members 451-1 (shown) and 452-2 (not shown) and is positioned to receive rotatable member 453 within a threaded lumen (not shown). Here, interface member 456 is configured generally as a nut to interface with the bolt-like rotatable member 453.

FIG. 18G is a cross-sectional perspective view of spacer 100 taken along line 18G-18G of FIG. 18E. Here, interface member 456 with threaded lumen 461 is shown in more detail. Interface member 456 also includes a transverse lumen 462 that extends through interface member 456 as well as arm portions 102 and 103 as shown here. Hinge members 452-1 and 452-2 are shown located within the upper portion 462-1 and lower portion 462-2 of lumen 462, respectively. Each hinge member 452 can be coupled with one of arm portion 102, arm portion 103 or interface member 456 to allow rotation of arm portions 102 and 103 around interface member 456. Arm portion 103 includes a recess 460 configured to allow pivoting of arm portion 103 around interface member 456. Arm portion 102 preferably includes a similar recess (not shown). As can be seen here, arm portions 102 and 103 are overlapping within the hinge region in a stacked fashion (the order in which arm portions 102 and 103 are stacked can be varied, including placement of each arm member only on opposing sides of interface member 456).

Rotatable member 453 is shown in cross-section as well with threaded lumen 454 visible therein. Rotatable member 453 is held in position with respect to U-shaped member 455 by the enlarged head portion of rotatable member 453 and the opposing retainer 463, which is configured here as a disk or washer. Retainer 463 can be securely fixed to rotatable member 453 in any desired manner (e.g., welding, through adhesives, soldering and the like). Retainer 463 has a function of at least holding rotatable member 453 in place while at the same time allowing rotation of member 453 with respect to U-shaped member 455.

FIG. 18H is a perspective view of an embodiment of delivery system 200 deploying the embodiments of spacer 100 described with respect to FIGS. 18A-G. Specifically, operation of system 200 with the embodiment described with respect to FIGS. 18C-G will be described here. Delivery system 200 can include a proximal handle 471 coupled with an elongate tubular shaft 474 that extends distally and is fixed to a shaft base housing 475. Shaft base housing 475 can be located at the base of the shaft (although not required) and is preferably configured to house the proximal portion of spacer 100 during the delivery procedure as shown here where spacer 100 is being placed between two adjacent spinous processes 14 of a patient's spinal column 10.

An arm control actuator 472 is coupled with proximal handle 47 1. Arm control actuator 472 is configured to rotate with respect to handle 471 and control the opening and closing of arm portions 102 and 103. An engagement control actuator 473 is also coupled with proximal handle 471 and is configured to control the engagement (e.g., release and re-engagement) of spacer 100 with delivery system 200. Engagement control actuator 473 can also be configured to translate distally and/or proximally. As shown here, each of actuators 472 and 473 and handle 471 can have any desired engagement features, such as ridges 476 and 477 on actuators 472 and 473, respectively, for increasing tactile control and feel for the user.

FIG. 18I is a perspective view of delivery system 200 without spacer 100. Shown here is proximal handle 471, arm control actuator 472, elongate shaft 474 and housing 475, which is located on the distal end of shaft 474. Housing 475 includes a open portion 481 configured to hold spacer 100 in a stable manner, preferably in a close or contacting fit. The distal end of an elongate spacer retainer 478 is shown protruding into recess 481 from within elongate shaft 474. Retainer 478 preferably has a threaded distal end that acts as a release mechanism (similar to release mechanism 245) for releasably interfacing with threaded lumen 454 of rotatable member 453. Also shown is the distal end of elongate tubular member 479 having a polygonal recessed interface 482 thereon. Interface 482 is configured to receive the proximal polygonal head of rotatable member 453. Both retainer 478 and elongate member 479 are rotatable independently of each other and elongate shaft 474 and housing 475. It should be noted that any other desired manner of engagement, other than a threaded and polygonal interfaces described here, can also be used.

FIG. 18J is a cross-sectional view of delivery system 200 with spacer 100 in an open configuration. This cross-section is taken along line 18J-18J of FIG. 18H (with spinal column 10 omitted). Within elongate shaft 474 is elongate member 479 the proximal end of which is fixedly coupled with arm control actuator 472. Within elongate tubular member 479 is elongate retainer 478 having the threaded distal end for interfacing with threaded lumen 454 (not shown). The proximal end of retainer 478 is fixedly coupled with engagement control actuator 473. A bias member 480, which in this embodiment is a spring, is located between actuator 472 and actuator 473. Bias member 480 maintains a reverse bias (or a bias in a proximal direction) on engagement control actuator 473. This has the effect of biasing spacer 100 and engagement control actuator 473 towards a proximal position. When spacer 100 is released from retainer 478, engagement control actuator 473 is free to move proximally from the bias exerted by bias member 480. This movement can provide tactile feedback to the user indicating that the spacer 100 has been fully released. Also, system 200 can be configured to provide other feedback, such as electronic visual or audio feedback, and the like, indicative of release of spacer 100, based either on the movement of actuator 473, loss of electrical contact with spacer 100, capacitive or magnetic sensors, and the like.

During deployment, the medical professional will preferably advance system 200 with spacer 100 in the open configuration into a desired position between adjacent spinous processes of the patient's spinal column. The medical professional can then rotate actuator 472 (either clockwise or counterclockwise) to close spacer 100. If, prior to release of spacer 100, the medical professional desires to re-position spacer 100, he or she can reverse the rotation of arm control actuator 472 to re-open spacer 100 and release from the interspinous tissue. Once in the desired new position, arm control actuator 472 can be used to close spacer 100 onto the interspinous tissue. Engagement control actuator 473 can then be advanced distally to free spacer 100 from housing 475, and subsequently (or simultaneously) rotated to release spacer 100 from delivery system 200. To do so without loosening rotatable member 453, which was tightened during the closure step, the winding of threaded distal end of retainer 479 is preferably counter that of rotatable member 453, such that the rotation of retainer 473 both releases spacer 100 and also tightens rotatable member 453. If the medical professional desires to re-engage spacer 100, then engagement control actuator 473 can be advanced distally to expose the distal end of retainer 478 and allow alignment with lumen 454 of rotatable member 453 on spacer 100. Engagement control actuator 473 can then be rotated to engage retainer 478 with lumen 454 and re-capture spacer 100.

FIG. 18K is a side view of this example embodiment of spacer 100 and shows raised upper surface 490-1 in greater detail. FIG. 18L is a cross-sectional view showing the right side of this example embodiment of spacer 100 in place adjacent a vertebral body 11 (surrounding soft tissue is not shown). Here, it can be seen that raised upper surface 490-2 closely fits against a corresponding ascending surface 31 at the base of spinous process 14. The base surface 32 of spinous process 11 extends over the relatively thick distal section of arm portion 103 and slightly into the open region 403. The base surface 32 transitions to the posterior (back-side) surface 33 adjacent the upper side of proximal member 455. The U-shaped proximal member 455 effectively fits around this posterior surface 33 and slides over the opposing lateral sides of spinous process 14. Together, U-shaped proximal member 455 and raised upper surfaces 490 act to provide a close fit with spinous process 14 and facilitate maintenance of spacer 100 in position relative to spinous process 14. Preferably, at least raised upper surface 490 is placed in close contact with spinous process 14 directly or by way of any intervening soft tissue. Variable surfaces, similar to raised upper surface 490, and other features can also be included on the lower side of spacer 100 to interface with the adjacent inferiorly located spinous process (not shown).

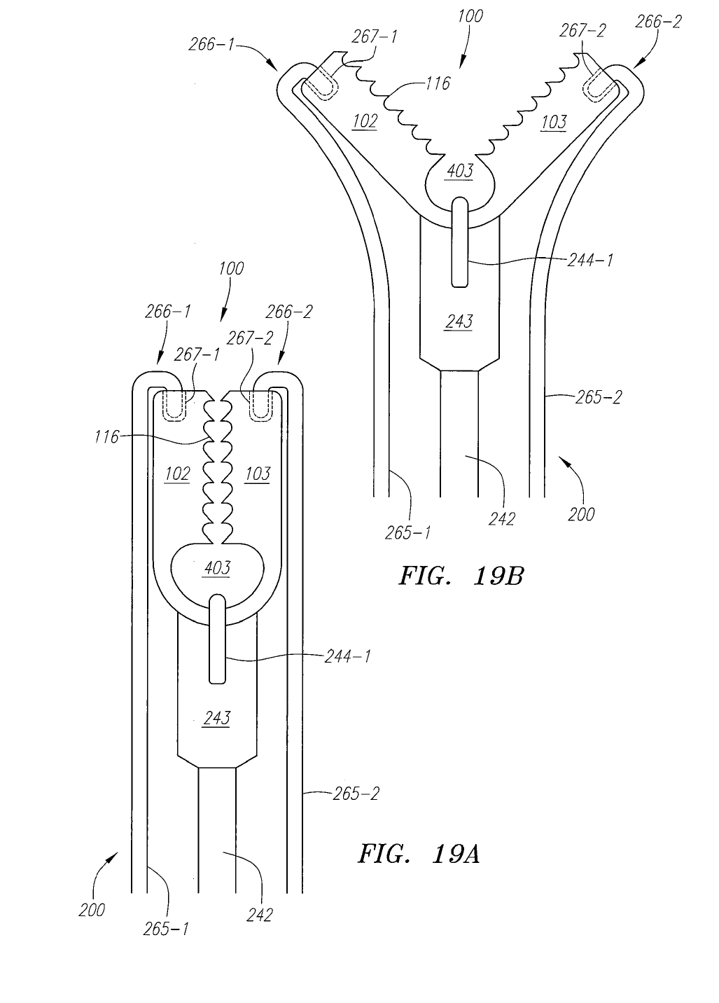

FIGS. 19A-B are top-down views of additional example embodiments of a spacer 100 and delivery system 200. Here, spacer 100 is biased towards the closed state depicted in FIG. 19A. Spacer 100 is deployable without the use of a closure body, although spacer 100 can be configured with a closure body if desired. Delivery system 200 includes elongate retraction members 265-1 and 265-2, each having a distally located grasping portion 266-1 and 266-2, respectively. Spacer 100 includes recesses 267-1 and 267-2 at or near the distal end of arm portions 102 and 103, respectively. Recesses 267 are preferably configured to interface with grasping portions 266, which is this embodiment are configured as hooks.

FIG. 19A shows the device in an at-rest state and FIG. 19B depicts spacer 100 after retraction members 265 have been retracted. This retraction causes arm portions 102 and 103 to be likewise retracted apart from each other, thereby opening spacer 100 for placement over the interspinous tissue. One of skill in the art will readily recognize that control of retraction members 265 can be integrated into actuator 202 (not shown) or by use of an additional actuator on delivery system 200. Spacer 100, in the configuration depicted in FIG. 19B, is positioned as desired, at which point retraction members 265 can be distally advanced to transition spacer 100 back to the closed state. In this manner, spacer 100 can be transitioned back and forth between the open and closed states as many times as it needed to complete proper positioning of the spacer and achieve proper deployment of the spacer. Alternatively, retraction members 265 can be removed to allow the bias of spacer 100 to force arm portions 102 and 103 back towards the closed state.

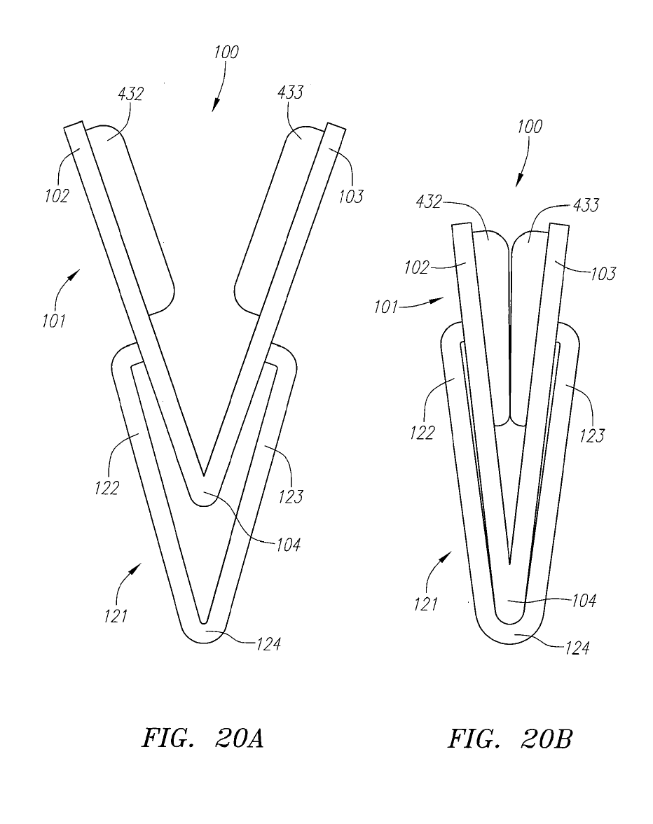

FIGS. 20A-B depict another example embodiment of spacer 100 having a main spacer body 101 and a closure body 121 in the open and closed states, respectively. Here, arm portions 102 and 103 are joined at connective portion 104, which is configured with a relatively angled or V-like configuration. Connective portion 124, which connects arm portions 122 and 123 of closure body 121, also preferably has a corresponding shape. The angled or V-like configuration facilitates the advancement of closure body 121 over main spacer body 101. The angled configuration can also make it easier to force arm portions 102 and 103 together to close spacer 100.

On the inner surfaces of arm portions 102 and 103 are relatively soft, cushion-like elements 432 and 433, respectively. Cushion-like elements 432 and 433 are configured to provide a less rigid interface with the interspinous tissue, which can reduce the risk of tearing the interspinous tissue and can allow accommodation of anatomies having relatively thin interspinous tissue. Cushion-like portions 432 and 433 can be formed from any compliant or conformable, flexible material, such as polymers and the like, and can have a solid construction or can include multi-component constructions, e.g., where an outer shell or membrane is filled with an inner gel or fluid. Portions 432 and 433 can also be configured to cover all of spacer 100 (or any portion thereof) to accommodate not only the interspinous tissue but also the spinous processes located superiorly and inferiorly to spacer 100.

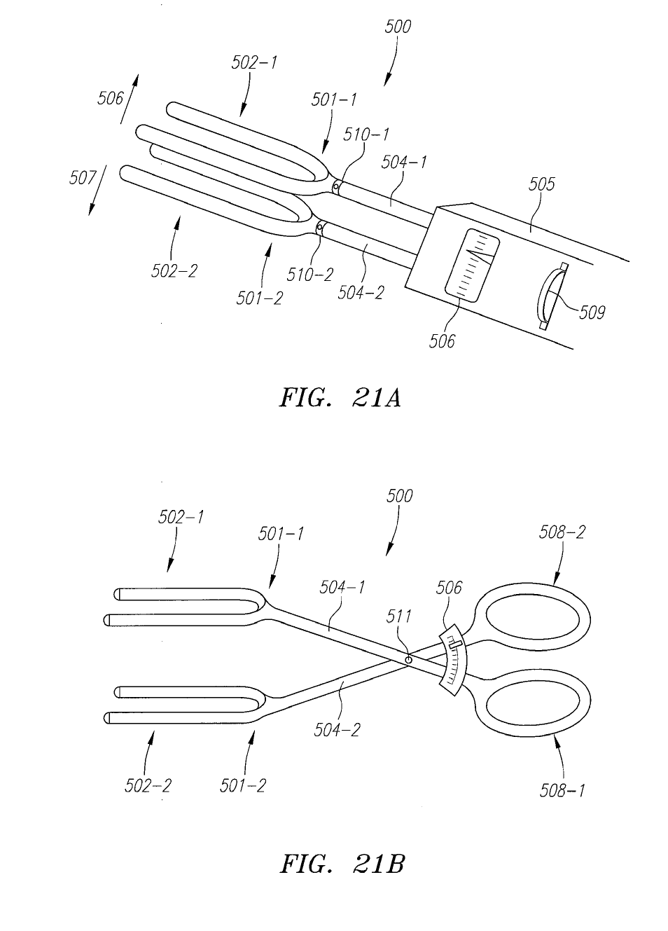

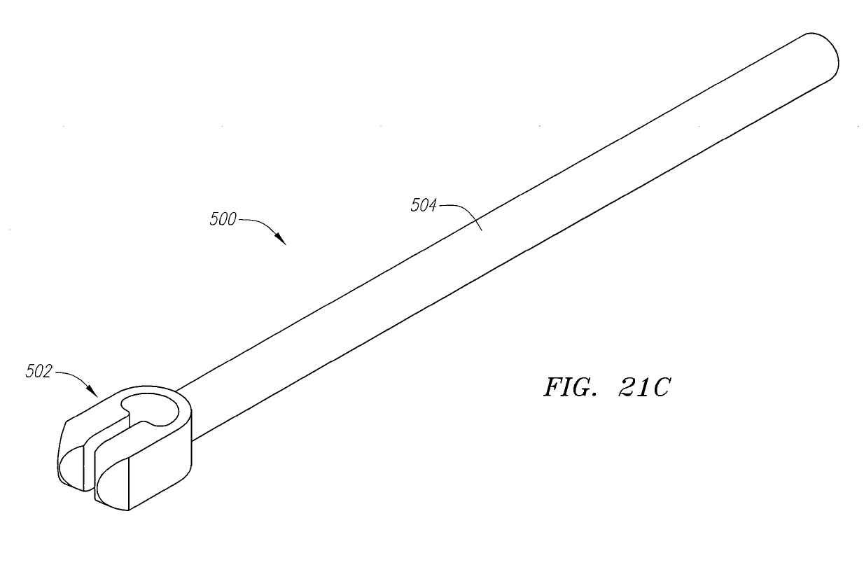

FIG. 21A depicts an example embodiment of a measurement tool 500 for use in measuring the distance between adjacent spinous processes for the purposes of providing sizing information for spacer 100. Tool 500 can also be configured to assess the profile of the space present between the adjacent spinous processes. Here, measuring tool 500 includes two measuring elements 501-1 and 501-2, each having a shaft 504 and a distal fork-like or U-shaped portion 502. Use of forked portions 502 allows measuring tool 500 to measure the interspinous space without the need for removal of interspinous tissue. Elements 501-1 and 501-2 can be moved in relation to each other in order to adequately measure the space between adjacent spinous processes.

For instance, while the patient is preferably in a state of slight flexion, tool 500 can be inserted between adjacent spinous processes in the location where spacer 100 is to be implanted and one or more of elements 501 can then be moved apart in directions 506 and 507 until the tissue surrounding the adjacent spinous processes are contacted by the fork-like portions 502-1 and 502-2 and further motion is prevented. Motion can also occur in the directions opposite 506 and 507. The elements 501 are coupled with a housing 505 that includes a guide 506 for displaying the distance between adjacent spinous processes and an actuator 509 for moving one or more of elements 501.

Assessment of the profile of this space between the adjacent spinous processes can be accomplished by tilting each fork-like portion 502 about hinges 510-1 and 510-2 until the fork-like portion 502 is generally flush against the tissue surrounding the respective spinous process (or the spinous process itself). The space between the opposing prongs of each fork-like portion 502 can also be made to be adjustable to accommodate various different anatomies and to ensure that an accurate measurement is performed. For instance, patients having unusually thick interspinous tissue may require the distance between the opposing prongs to be increased. Thus, the incorporation of a hinge and latching mechanism at the base of each forked portion can be desired.

FIG. 21B depicts another example embodiment of a measurement tool 500 for use in measuring the distance between adjacent spinous processes for the purposes of providing sizing information for spacer 100. Here, tool 500 has a scissor-like configuration with both measuring elements 501-1 and 501-2 pivotably coupled together by hinge 511. Handle portions 508-1 and 508-2 are at the proximal ends of each measuring element 501-1 and 501-2, respectively. The medical professional can insert tool 500 between adjacent spinous process and move forked portions 502-1 and 502-2 apart until contact with each spinous process is achieved. Reference to gauge 506 provides the measurement information that can be used in selecting the appropriately sized spacer.

FIG. 21C depicts another example embodiment of a measurement tool 500 for use in sizing spacer body 101. Here, tool 500 includes shaft 504 and a rigidly fixed distal forked portion 502 which resembles spacer body 101. A set of multiple tools 500 can be provided to the medical professional where each tool 500 has a different sized forked portion 502 corresponding to the various different sizes of spacer body 101 that are provided. Iterative use of the different measurement tools 500 allows the medical professional to determine the appropriately sized spacer 101 based on the desired fit of forked portion 502 over the interspinous tissue and between the adjacent spinous processes.

It should be noted that various embodiments are described herein with reference to one or more numerical values. These numerical value(s) are intended as examples only and in no way should be construed as limiting the subject matter recited in any claim, absent express recitation of a numerical value in that claim.

While the embodiments are susceptible to various modifications and alternative forms, specific examples thereof have been shown in the drawings and are herein described in detail. It should be understood, however, that these embodiments are not to be limited to the particular form disclosed, but to the contrary, these embodiments are to cover all modifications, equivalents, and alternatives falling within the spirit of the disclosure. Statements expressly indicating that certain features are not limited in a particular manner should not be interpreted as implying that the absence of such statements with regard to other features implies that those other features are in any way limited to the disclosed embodiment.

What is claimed is:

1. An interspinous spacer, comprising:
a first spacing portion configured for spacing a pair of adjacent spinous processes by placement on a first side of an interspinous ligament located between the adjacent spinous processes;
a second spacing portion configured for spacing the pair of adjacent spinous processes by placement on a second side of the interspinous ligament located between the adjacent spinous processes; and
a connective portion coupling the first and second spacing portions together, wherein the connective portion is configured for placement on a posterior side of a supraspinous ligament located between the pair of adjacent spinous processes, the spacer having no load-bearing, spacing portion configured for placement through the portion of the interspinous ligament located between the pair of adjacent spinous processes.

2. The interspinous spacer of claim 1, further comprising a tissue anchor configured to pierce entirely through the portion of the interspinous ligament from the first side to the second side with only insubstantial injury to the interspinous ligament

3. The interspinous spacer of claim 1, wherein the spacer has a general U-shape.

4. The interspinous spacer of claim 1, wherein the first and second spacing portions and connective portion are on a main spacer body, the spacer further comprising a closure body configured to couple with the main spacer body.

5. The interspinous spacer of claim 1, wherein the first and second spacing portions each comprise a tissue-engagement feature configured to engage with the interspinous ligament.

6. The interspinous spacer of claim 1, further comprising a proximally located open region between the first and second spacing portions, the open region having a width that is relatively greater than a width between the first and second spacing portions in the closed state.

7. The interspinous spacer of claim 1, wherein the first and second spacing portions are deflectable from an open state to a closed state where the first and second spacing portions are spaced relatively closer together.

8. The interspinous spacer of claim 7, wherein the first and second spacing portions are biased to deflect towards the closed state.

9. The interspinous spacer of claim 1, wherein the first and second spacing portions are fixed in relation to each other.

10. The interspinous spacer of claim 1, wherein the first and second spacing portions are pivotably coupled with the connective portion.

11. The interspinous spacer of claim 1, wherein the first spacing portion and the second spacing portion are moveable between an open and closed configuration, the spacer further comprising a proximal member moveably coupled with the first and second spacing portions, wherein movement of the proximal member with respect to the spacing portions causes the spacing portions to transition between the open and closed configurations.

12. The interspinous spacer of claim 11, further comprising a proximally located open region between the first and second spacing portions, the open region having a width that is relatively greater than a width between the first and second spacing portions in the closed state.

13. The interspinous spacer of claim 12, further comprising an actuator configured to move the proximal member in relation to the first and second spacing portions.

14. The interspinous spacer of claim 13, wherein the spacer further comprises an interface member coupled with the first and second spacing portions and configured to couple with the actuator, the interface member being pivotable with respect to both the first and second spacing portions.

15. The interspinous spacer of claim 14, wherein the interface member comprises a threaded lumen configured to interface with a threaded surface of the actuator and wherein the actuator is coupled with the proximal member such that the actuator can be rotated with respect to the proximal member.

16. The interspinous spacer of claim 15, wherein the proximal member is configured as a U-shaped strut residing over at least a proximal portion of both the first and second spacing portions.

17. The interspinous spacer of claim 16, wherein the U-shaped strut is configured to slide over opposing lateral sides of a spinous process.

18. The interspinous spacer of claim 1, wherein the first and second spacing portions are each configured as elongate arm-like struts.

19. An interspinous spacer, comprising:
a first spacing portion configured for spacing a pair of adjacent spinous processes by placement on a first side of a portion of an interspinous ligament located between the adjacent spinous processes;
a second spacing portion configured for spacing the pair of adjacent spinous processes by placement on a second side of the portion of the interspinous ligament located between the adjacent spinous processes; and
an attachment mechanism coupling the first and second spacing portions together, wherein the attachment mechanism is configured for placement through the interspinous tissue between the pair of adjacent spinous processes, the attachment mechanism having no load-bearing, spacing portion configured as the physical barrier to compressive movement between the pair of adjacent spinous processes.

20. A method for implanting a spacer into the body of a human patient, comprising:
creating an access opening in the back of a patient;
positioning a spacer over a portion of the interspinous ligament between adjacent spinous processes without creating a prior incision through the portion of the interspinous ligament, wherein positioning comprises positioning a first spacing portion on a first side of the interspinous ligament and a second spacing portion on a second, opposite side of the interspinous ligament;
closing the spacer over the interspinous ligament by moving the first and second spacing portions towards each other; and
closing the access opening.

21. The method of claim 20, wherein, prior to closing the access opening, the spacer is fully deployed in the back of the patient without creating any opening entirely through the interspinous ligament.

22. The method of claim 20, wherein, after closing the spacer, the spacer is fully deployed in the back of the patient without creating any opening partially into the interspinous ligament.

23. The method of claim 21, wherein, after closing the spacer, the spacer is fully deployed in the back of the patient without creating any opening partially into the supraspinous ligament.
